(19) United States
(12) Patent Application Publication
(10) Pub. No.: US 2025/0059160 A1
(43) Pub. Date: Feb. 20, 2025

(54) Chemical Activators of Nicotinamide Mononucleotide adenylyltransferase 2 (NMNAT2) and Uses Thereof

(71) Applicant: Tsinghua University, Beijing City (CN)

(72) Inventors: Gelin Wang, Beijing City (CN); Yefeng Tang, Beijing City (CN); Ruoxi Zhang, Beijing City (CN); Leibo Wang, Beijing City (CN); Shuangquan Chen, Beijing City (CN)

(21) Appl. No.: 18/938,755

(22) Filed: Nov. 6, 2024

Related U.S. Application Data
(63) Continuation-in-part of application No. PCT/CN2018/117723, filed on Nov. 27, 2018.

(51) Int. Cl. C07D 401/12; A61K 31/17; A61K 31/341; A61K 31/381; A61K 31/397; A61K 31/4025; A61K 31/4402; A61K 31/4427; A61K 31/443; A61K 31/4436; A61K 31/444; A61K 31/4545; C07C 335/40; C07D 205/04; C07D 213/59; C07D 307/54; C07D 333/24; C07D 403/12; C07D 405/12; C07D 409/12; C12Q 1/32; C12Q 1/48; G01N 21/77

(52) U.S. Cl. CPC C07D 401/12; A61K 31/17; A61K 31/341; A61K 31/381; A61K 31/397; A61K 31/4025; A61K 31/4402; A61K 31/4427; A61K 31/443; A61K 31/4436; A61K 31/444; A61K 31/4545; C07C 335/40; C07D 205/04; C07D 213/59; C07D 307/54; C07D 333/24; C07D 403/12; C07D 405/12; C07D 409/12; C12Q 1/32; C12Q 1/48; G01N 2021/7786; G01N 2333/904; G01N 2333/91245

(57) ABSTRACT

The present disclosure relates to novel NMNAT2 activators, semicarbazones and thiosemicarbazones, to processes for preparing them, to pharmaceutical preparations comprising them, to the method by administering the novel semicarbazones and thiosemicarbazones for the treatment and/or prevention of diseases and to the use thereof for the production of a medicament for the treatment and/or prevention of diseases, especially neurodegenerative and age-associated diseases or conditions associated with NAD loss. The present disclosure also provides a method for high throughput screening of NMNAT2 activators.

### RELATED APPLICATIONS

The present application is a continuation-in-part application of International Patent Application No. PCT/CN2018/117723, filed on Nov. 27, 2018, which is hereby incorporated by reference in its entirety.

### TECHNICAL FIELD

The present disclosure relates to novel NMNAT2 activators, semicarbazones and thiosemicarbazones, to processes for preparing them, to pharmaceutical preparations comprising them, to a method for treating and/or preventing diseases by comprising administering the novel semicarbazones and thiosemicarbazones to a subject in need thereof, and to the use thereof for the production of a medicament for the treatment and/or prevention of diseases, especially neurodegenerative and age-associated diseases or conditions associated with NAD loss.

### BACKGROUND

Nicotinamide mononucleotide adenylyltransferase (NMNAT) is required for NAD biosynthesis. NAD is synthesized via de novo production from tryptophan, the Preiss-Handler pathway from nicotinic acid (NA), and salvage pathways from nicotinamide (NAM), or nicotinamide riboside (NR) (FIG. 1). Among them, mammalian cells appear to rely mainly on the salvage pathway that recycles the NAM generated by NAD-consuming enzymes. The regulatory enzyme Nicotinamide phosphoribosyltransferase (NAMPT) converts NAM to nicotinamide mononucleotide (NMN), followed by production of NAD via the NMNAT enzyme directly. In a second salvage pathway, NR from food source or intracellular metabolite, is incorporated into NAD via nicotinamide riboside kinases (NRKs) and NMNAT. The Preiss-Handler pathway is initiated by nicotinate phosphoribosyltransferase (NAPRT) to use NA to form NAMN, which is sequentially converted to NAD by NMNAT and NAD synthetase. In the de novo pathway, tryptophan is first transformed via several steps into NAMN, which is also converted to NAD via NMNAT and NAD synthetase. As such NMNAT is necessary for all the NAD biosynthetic pathways.

There are three enzyme isoforms of NMNAT that are differentially localized, namely, NMNAT1 in nucleus; NMNAT2 in Golgi; NMNAT3 in mitochondria, suggesting that NAD biosynthesis is compartmentalized within the cell (Cambronne et al., 2016) (Ryu et al., 2018). Despite that mutations in NMNAT1 were found to cause Leber congenital amaurosis with early-onset severe macular and optic atrophy (Perrault et al., 2012), the biological importance and the regulation of this compartment-specific NAD synthesis is poorly understood.

SARM1 was identified in a forward genetic screen in Drosophila as a key mediator of axon death pathway. Activation of SARM1 promotes cellular NAD depletion and subsequent axon degeneration. The connection between NAD biosynthetic pathway and axon degeneration pathway is strongly supported by the evidence that the lethality of NMNAT2-deficiency in mice is rescued by deletion of the Sarm1 gene.

Up to now, there is no reported chemical that can activate NMNAT, the essential enzyme for all the NAD biosynthetic pathways.

### SUMMARY

The present inventors have found novel semicarbazones and thiosemicarbazones, which are chemical activators of Nicotinamide mononucleotide adenylyltransferase (NMNAT).

The present disclosure provides compounds of the general formula

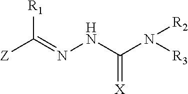

in which
Z represents a heteroaryl group, an aryl group or a C₁-C₆ alkyl group;
R₁ represents hydrogen, a C₁-C₆ alkyl group, a C₃-C₆ cycloalkyl group, or an aryl group;
X represents Se, NH, O or S; and
R₂ or R₃ represents hydrogen, a —OR₄ group, a C₁-C₆ alkyl group, a C₃-C₆ alkenyl group, a C₅-C₈ cycloalkenyl group, a (C₁-C₆ alkyl)-(C₅-C₈ cycloalkenyl) group, a C₃-C₆ alkynyl group, a pyrrolidinyl group, a (C₁-C₆ alkyl)-pyrrolidinyl group, a piperidinyl group, a (C₁-C₆ alkyl)-piperidinyl group, a morpholinyl group, a (C₁-C₆ alkyl)-morpholinyl group, a piperazinyl group, a (C₁-C₆ alkyl)-piperazinyl group, a C₃-C₆ cycloalkyl group, —NR₅R₆, a heteroaryl group, an aryl group, a (C₁-C₆ alkyl)-heteroaryl group, a (C₁-C₆ alkyl)-aryl group, a (C₁-C₆ alkyl)-CO—R₇ group, a (C₁-C₆ alkyl)-NR₅R₆ group, or a (C₁-C₆ alkyl)-OR₄ group; or

R₂ and R₃ together with the nitrogen atom to which they are attached form a 4-to 6-membered heterocycloalkyl;
in which R₄ represents hydrogen, a C₁-C₆ alkyl group, a (C₁-C₆ alkyl)-heteroaryl group or a (C₁-C₆ alkyl)-aryl group;
in which R₅ or R₆ represents hydrogen, a C₁-C₆ alkyl group, a carbonyl group, a sulfoxide group or a sulfone group;
in which R₇ is a —O—(C₁-C₆ alkyl) group, an amino group, a —NH—(C₁-C₆ alkyl) group or a —NH—N═CR₈-heteroaryl group;
   in which R₈ is a C₁-C₆ alkyl group;
      and the diastereomers, enantiomers, metabolites, salts, solvates thereof or solvates of the salts thereof.

The present compounds are especially suitable for treatment and/or prevention of neurodegeneration and age-associated diseases or conditions associated with NAD loss. Particular mention should be made here of amyotrophic lateral sclerosis, Parkinson's disease, traumatic brain injury, neonatal nerve crush injury, Alzheimer's disease, Chemotherapy-induced peripheral neuropathy (CIPN), ischemia, retinal degeneration, age-associated deficiency of neurogenesis, hypoadiponectinemia, and multi-organ insulin resistance.

The present disclosure provides a pharmaceutical preparation which comprises at least one compound according to the disclosure, typically together with one or more inert, nontoxic, pharmaceutically suitable excipients, and the use thereof for the aforementioned purposes. The present disclosure provides a method for preparing the present compound, comprising scheme 5 and any of schemes 1 to 4:

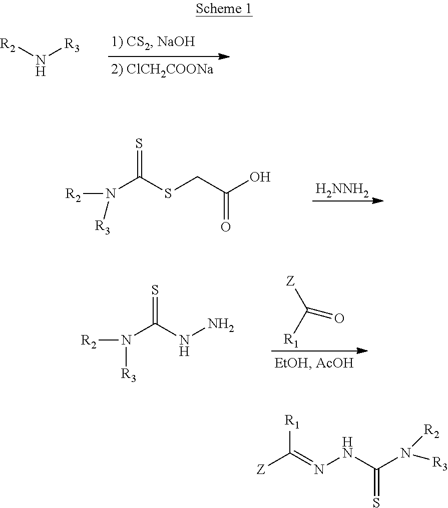

in which groups Z, R₁, R₂, and R₃ are defined above;

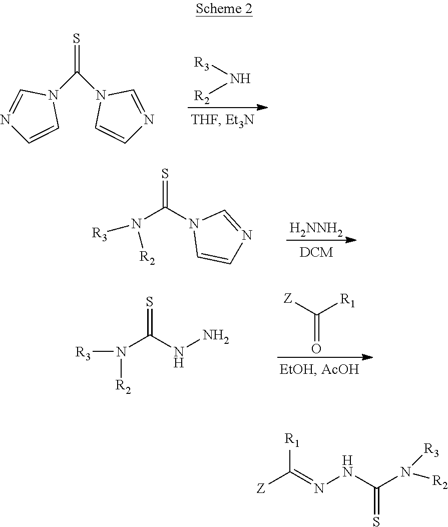

in which groups Z, R₁, R₂, and R₃ are defined above;

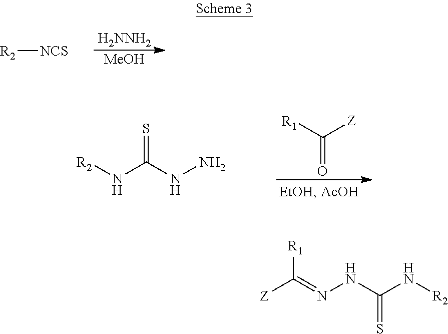

in which groups Z, R₁, and R₂ are defined above;

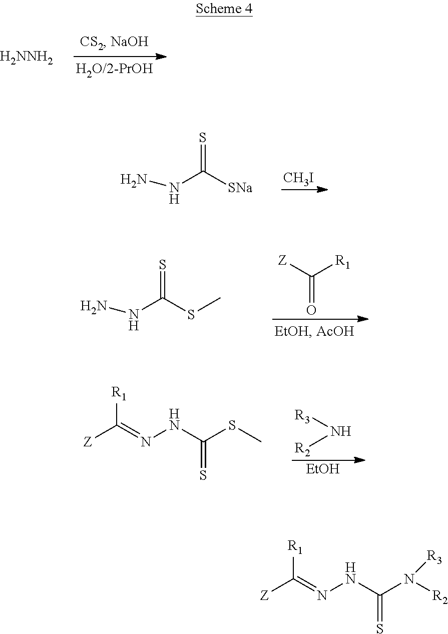

in which groups Z, R₁, R₂, and R₃ are defined above;

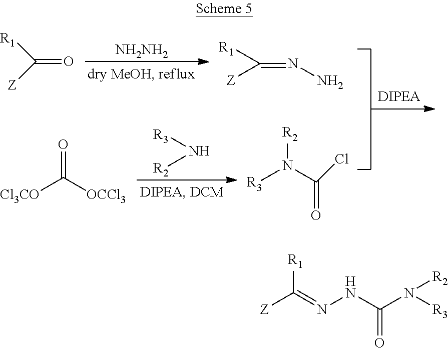

in which groups Z, R₁, R₂, and R₃ are defined above.

The present disclosure provides a method for high throughput screening of NMNAT2 activators, comprising
(a) adding compounds to NMNAT2 enzyme reaction mixture;
(b) initiating NMNAT2 enzyme reaction by adding NMN;
(c) monitoring the conversion of NMN to NADH by measuring fluorescence intensity of NADH at an excitation of 340 nm and an emission of 445 nm (Y axis) as a function of time (X axis), and then calculating reaction rate;
(d) obtaining relative NMNAT2 enzyme reaction rate in the presence of each compound by normalizing with DMSO-treated control; and
(e) selecting compounds with the relative reaction rate higher than 120% as NMNAT2 activators.

### BRIEF DESCRIPTION OF THE DRAWINGS

FIG. 1 shows NAD biosynthetic pathways, in which NAD is synthesized via de novo production from tryptophan, and salvage pathways from nicotinamide (NAM), nicotinic acid (NA) or nicotinamide riboside (NR).

FIG. 2 shows NMNAT2 activation by compound NSC. In a doubly coupled enzyme assay, the first enzyme NMNAT2 (it converted NMN to NAD) and the second enzyme ADH (it converted NAD to NADH) as well as compound NSC at a concentration of 3 μM were used, and the conversion of NMN to NADH was monitored by measuring fluorescence intensity of NADH at an excitation of 340 nm and an emission of 445 nm (Y axis) as a function of time (X axis).

FIG. 3 shows that compound NSC activates NMNAT2 and NMNAT1, but not NMNAT3. A doubly coupled enzyme assay was prepared for NMNAT1, NMNAT2 and NMNAT3. NMNAT activation is normalized, where the reaction rate of NMNAT in the presence of 3 μM of compound NSC was compared to that of a DMSO-treated control.

FIG. 4 shows that compound NSC protects cultured cells against SARM1-mediated toxicity. SARM1-expressing cells were treated with compound NSC at 0.1, 0.3, 1, 3, 10, and 30 μM. After 24 hours, the cell images in bright field were taken under the microscope before cell viability was determined by measuring ATP levels using CellTiter-Glo assay kit.

FIG. 5 shows that compound 20 protects against paclitaxel-induced periphery neuropathy in vivo. The procedure of CIPN mouse model and treatment regimen is shown in the upper panel. In this CIPN mice model, compound 20 (6 mg/kg/day i.p.) significantly increased the mice paw pressure threshold (lower panel).

FIG. 6 shows compound NSC protects against paclitaxel-induced periphery neuropathy in vivo. In this CIPN mice model, compound NSC significantly increased the mice paw pressure threshold.

FIG. 7 shows activities of compounds NSC and 1-40 in an in vitro enzyme assay measuring activation of NMNAT2 and a cell-based assay measuring the degree of protection from SARM1 expression in U2OS cells. In the first assay, the compounds were added at 1, 3, 10, 30 and 100 μM in the reaction mixture. The reaction rate of NMNAT2 enzyme was calculated as the slope of the F_{340/445} versus time curve, and was normalized by DMSO-treated control. The dose response curves were plotted to assess the effects of individual compounds on NMNAT2 enzymatic activity. In the second assay, SARM1-expressing cells were treated with compounds NSC and 1-40 at 0.1, 0.3, 1, 3, 10, and 30 μM. After 24 hours, the cell survival was measured by Cell-titer Glo kit, and normalized by DMSO-treated control. The dose response curves were plotted to evaluate the cell-protecting activity of the compounds.

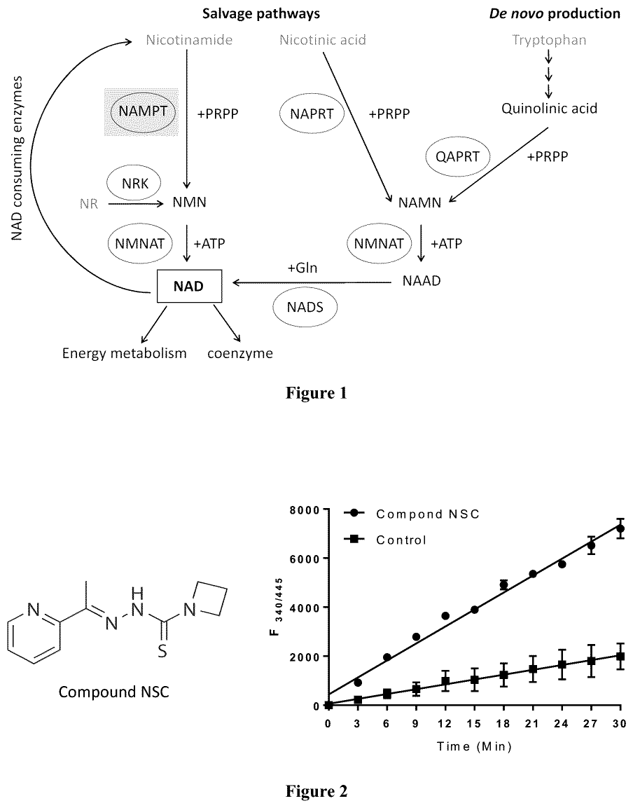

### DETAILED DESCRIPTION

Compounds of the disclosure are the compounds of the formula (I) and the salts, solvates and solvates of the salts thereof, the compounds that are encompassed by formula (I) and are of the formulae mentioned below and the salts, solvates and solvates of the salts thereof and the compounds that are encompassed by formula (I) and are cited below as working examples and the salts, solvates and solvates of the salts thereof if the compounds that are encompassed by formula (I) and are mentioned below are not already salts, solvates and solvates of the salts.

Preferred salts in the context of the present disclosure are physiologically acceptable salts of the compounds according to the disclosure. Nevertheless, the disclosure also encompasses salts which themselves are unsuitable for pharmaceutical applications but which can be used, for example, for the isolation or purification of the compounds according to the disclosure.

Physiologically acceptable salts of the compounds according to the disclosure include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the disclosure also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine. Solvates in the context of the disclosure are described as those forms of the compounds according to the disclosure which form a complex in the solid or liquid state by coordination with solvent molecules. Hydrates are a specific form of the solvates in which the coordination is with water.

The compounds according to the disclosure may, depending on their structure, exist in different stereoisomeric forms, i.e. in the form of configurational isomers or else, if appropriate, as conformational isomers (enantiomers and/or diastereomers, including those in the case of atropisomers). The present disclosure therefore encompasses the enantiomers and diastereomers, and the respective mixtures thereof. The stereoisomerically homogeneous constituents can be isolated from such mixtures of enantiomers and/or diastereomers in a known manner; chromatographic processes are preferably used for this purpose, especially HPLC chromatography on an achiral or chiral phase.

If the compounds according to the disclosure can occur in tautomeric forms, the present disclosure encompasses all the tautomeric forms.

The present disclosure also encompasses all suitable isotopic variants of the compounds according to the disclosure. An isotopic variant of a compound of the disclosure is understood here to mean a compound in which at least one atom within the compound of the disclosure has been exchanged for another atom of the same atomic number, but with a different atomic mass from the atomic mass which usually or predominantly occurs in nature. Examples of isotopes which can be incorporated into a compound according to the disclosure are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the disclosure, such as, in particular, those in which one or more radioactive isotopes have been incorporated, may be beneficial, for example, for the examination of the mechanism of action or of the active ingredient distribution in the body; because of the comparative ease of preparation and detectability, particularly compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds according to the disclosure may therefore in some cases also constitute a preferred embodiment of the present disclosure. Isotopic variants of the compounds according to the disclosure can be prepared by the processes known to those skilled in the art, for example by the methods described further below and the procedures described in the working examples, by using corresponding isotopic modifications of the respective reagents and/or starting compounds.

The present disclosure further provides all the possible crystalline and polymorphous forms of the compounds according to the disclosure, where the polymorphs may be present either as single polymorphs or as a mixture of a plurality of polymorphs in all concentration ranges.

The present disclosure additionally also encompasses prodrugs of the compounds according to the disclosure. The term “prodrugs” in this context refers to compounds which may themselves be biologically active or inactive but are reacted (for example metabolically or hydrolytically) to give compounds according to the disclosure during their residence time in the body. In the context of the present disclosure, unless specified otherwise, the substituents have the following meanings: Alkyl in the context of the disclosure is a straight-chain or branched alkyl radical having the particular number of carbon atoms specified. Examples of C₁-C₆ alkyl groups include methyl, ethyl, n-propyl, isopropyl, n-butyl, isobutyl, 1-methylpropyl, 2-methylpropyl, tert-butyl, n-pentyl, 1-ethylpropyl, 1-methylbutyl, 2-methylbutyl, 3-methylbutyl, 2,2-dimethylpropyl, n-hexyl, 1-methylpentyl, 2-methylpentyl, 3-methylpentyl, 4-methylpentyl, 1-ethylbutyl and 2-ethylbutyl. Preference is given to methyl, ethyl, n-propyl, isopropyl, n-butyl, tert-butyl, 2-methylbutyl, 3-methylbutyl and 2,2-dimethylpropyl. Particular preference is given to methyl, ethyl, propyl and tert-butyl.

Alkenyl in the context of the disclosure is understood to mean a straight-chain or branched, monovalent hydrocarbon radical having one or two C═C double bonds, for example, ethenyl, prop-1-enyl, prop-2-enyl, allyl (prop-1-enyl), allenyl, buten-1-yl or buta-1,3-dienyl radical. Preference is given to prop-2-enyl.

Cycloalkyl in the context of the disclosure is a monocyclic saturated alkyl radical having the number of carbon atoms specified in each case. Examples include cyclopropyl, cyclobutyl, cyclopentyl, and cyclohexyl. Preference is given to cyclopropyl and cyclohexyl, more preferably cyclopropyl.

The term “4- to 6-membered heterocycloalkyl” refers to a monocyclic saturated heterocycle having a total of 4 to 6 ring atoms, in which one or two ring carbon atoms are replaced by identical or different heteroatoms from the group of N, O and S; the heterocycloalkyl group may be bonded to the rest of the molecule via any one of the carbon atoms or, if present, a nitrogen atom.

The heterocycloalkyl group may, although this is not intended to constitute a restriction, for example, be a 4-membered ring such as azetidinyl, oxetanyl or thietanyl; or a 5-membered ring such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl; or a 6-membered ring such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl.

Preference is given to 4- or 6-membered heterocycloalkyl. Particular preference is given to azetidinyl and piperidinyl.

The term “aryl” is understood to mean a monovalent monocyclic aromatic ring system which has 6 ring atoms. Examples of aryl groups include phenyl and hydroxyphenyl group, preferably phenyl and 2-hydroxyphenyl group, more preferably phenyl.

The term “heteroaryl” is understood to mean a monovalent monocyclic aromatic ring system which has 5 or 6 ring atoms and contains at least one ring heteroatom and optionally one, two or three further ring heteroatoms from the group of N, O and S, and which is bonded to the rest of the molecule via a ring carbon atom or optionally (if the valency allows it) via a ring nitrogen atom.

Examples of 5-membered heteroaryl groups (“5-membered heteroaryl”) include thienyl, furyl, pyrrolyl, oxazolyl, thiazolyl, imidazolyl, pyrazolyl, isoxazolyl, isothiazolyl, oxadiazolyl, triazolyl, thiadiazolyl or tetrazolyl. Examples of 6-membered heteroaryl groups (“6-membered heteroaryl”) include pyridyl, pyridazinyl, pyrimidinyl, pyrazinyl or triazinyl.

In general, and unless stated otherwise, the heteroaryl radicals include all possible isomeric forms, for example tautomers and positional isomers in relation to the attachment point to the rest of the molecule. For example, the term “pyridyl” includes pyridin-2-yl, pyridin-3-yl and pyridin-4-yl. A further illustrative example is the term “thiazolyl”, which includes 1,3-thiazol-4-yl, 1,3-thiazol-5-yl and 1,3-thiazol-2-yl. Said examples are cited for illustration of the definition and are in no way to be understood as a limitation to the terms mentioned.

Preferred 5-membered heteroaryl are thienyl group, pyrrolyl group, and furyl group. Particular preference is given to thiophen-2-yl group, pyrrol-2-yl group, and furan-2-yl group.

Preferred 6-membered heteroaryl is pyridyl. Particular preference is given to pyridin-2-yl.

A symbol * at a bond denotes the bonding site in the molecule.

When radicals in the compounds according to the disclosure are substituted, the radicals may be mono- or polysubstituted, unless specified otherwise. In the context of the present disclosure, all radicals which occur more than once are defined independently of one another. Substitution by one, two or three identical or different substituents is preferred.

Particular embodiments of the present disclosure are compounds of the general formula in which
Z represents pyridyl group, thienyl group, pyrrolyl group, furyl group, hydroxyphenyl group, or a C₁-C₆ alkyl group;
R₁ represents hydrogen, a C₁-C₆ alkyl group, a C₃-C₆ cycloalkyl group, or phenyl group; X represents Se, NH, O or S; and
R₂ or R₃ represents hydrogen, a —OR₄ group, a C₁-C₆ alkyl group, a C₃-C₆ alkenyl group, a C₅-C₈ cycloalkenyl group, a (C₁-C₆ alkyl)-(C₅-C₈ cycloalkenyl) group, a C₃-C₆ alkynyl group, a pyrrolidinyl group, a (C₁-C₆ alkyl)-pyrrolidinyl group, a piperidinyl group, a (C₁-C₆ alkyl)-piperidinyl group, a morpholinyl group, a (C₁-C₆ alkyl)-morpholinyl group, a piperazinyl group, a (C₁-C₆ alkyl)-piperazinyl group, a C₃-C₆ cycloalkyl group, —NR₅R₆, pyridyl group, furyl group, phenyl group, a (C₁-C₆ alkyl)-pyridyl group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OR₄ group; or
R₂ and R₃ together with the nitrogen atom to which they are attached form a 4- to 6-membered heterocycloalkyl;
   in which R₄ represents hydrogen, a C₁-C₆ alkyl group, a (C₁-C₆ alkyl)-pyridyl group or a (C₁-C₆ alkyl)-phenyl group;
   in which R₅ or R₆ represents hydrogen, a C₁-C₆ alkyl group, a carbonyl group, a sulfoxide group or a sulfone group;
   in which R₇ is a —O—(C₁-C₆ alkyl) group, an amino group, a —NH—(C₁-C₆ alkyl) group or a —NH—N—CR₈-pyridyl group;
      in which Re is a C₁-C₆ alkyl group;
         and the diastereomers, enantiomers, metabolites, salts, solvates thereof or solvates of the salts thereof.

Particular embodiments of the present disclosure are compounds of the general formula in which
Z represents pyridyl group, thienyl group, pyrrolyl group, furyl group, hydroxyphenyl group, or a C₁-C₆ alkyl group;

R₁ represents hydrogen, a C₁-C₆ alkyl group, a C₃-C₆ cycloalkyl group, or phenyl group;
X represents O or S; and
R₂ or R₃ represents hydrogen, a C₁-C₆ alkyl group, a C₃-C₆ alkenyl group, a C₃-C₆ cycloalkyl group, —NR₅R₆, pyridyl group, furyl group, phenyl group, a (C₁-C₆ alkyl)-pyridyl group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OH group; or
R₂ and R₃ together with the nitrogen atom to which they are attached form a 4- to 6-membered heterocycloalkyl;
   in which R₅ or R₆ represents hydrogen or a C₁-C₆ alkyl group;
   in which R is a —O—(C₁-C₆ alkyl) group or a —NH—N═CR₈-pyridyl group;
      in which R₈ is a C₁-C₆ alkyl group;
         and the diastereomers, enantiomers, metabolites, salts, solvates thereof or solvates of the salts thereof.

Additional more particular embodiments of the present disclosure are compounds of the general formula in which
Z represents pyridyl group, thienyl group, pyrrolyl group, furyl group, hydroxyphenyl group, or
a C₁-C₆ alkyl group;
R₁ represents hydrogen, a C₁-C₆ alkyl group, a C₃-C₆ cycloalkyl group, or phenyl group;
X represents O or S; and
R₂ and R₃ represents hydrogen, a C₁-C₆ alkyl group; or either of R₂ and R₃ represents H, the other of R₂ and R₃ represents a C₁-C₆ alkyl group, a C₂-C₆ alkenyl group, a C₃-C₆ cycloalkyl group, —NR₅R₆, pyridyl group, furyl group, phenyl group, a (C₁-C₆ alkyl)-pyridyl group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OH group; or
R₂ and R₃ together with the nitrogen atom to which they are attached form a 4- to 6-membered heterocycloalkyl;
   in which R₇ is —-O-(C₁-C₆ alkyl) group or a —NH—N═CR₈-pyridyl group;
      in which R₈ is a C₁-C₆ alkyl group;
         and the diastereomers, enantiomers, metabolites, salts, solvates thereof or solvates of the salts thereof.

Especially more particular embodiments of the present disclosure are compounds of the general formula in which
Z represents pyridin-2-yl group, pyridin-3-yl group, pyridin-4-yl group, thiophen-2-yl group, pyrrol-2-yl group, furan-2-yl group, hydroxyphenyl group, or a C₁-C₆ alkyl group;
R₁ represents hydrogen, a C₁-C₆ alkyl group, a C₃-C₆ cycloalkyl group, or phenyl group; X represents O or S; and
R₂ and R₃ represents hydrogen, or a C₁-C₆ alkyl group; or
either of R₂ and R₃ represents H, the other of R₂ and R₃ represents a C₁-C₆ alkyl group, a C₂-C₆ alkenyl group, a C₃-C₆ cycloalkyl group, —NR₅R₆, pyridin-2-yl group, pyridin-3-yl group, furan-2-yl group, phenyl group, a (C₁-C₆ alkyl)-(pyridin-2-yl) group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OH group; or
R₂ and R₃ together with the nitrogen atom to which they are attached form a 4- to 6-membered heterocycloalkyl;
   in which R₇ is a —O—(C₁-C₆ alkyl) group or a —NH—N═CR₈-(pyridin-2-yl) group;
      in which R₈ is a C₁-C₆ alkyl group;
         and the diastereomers, enantiomers, metabolites, salts, solvates thereof or solvates of the salts thereof.

In a particular embodiment of the present disclosure, Z represents pyridyl group, thienyl group, pyrrolyl group, furyl group, hydroxyphenyl group, or a C₁-C₆ alkyl group; more particularly, Z represents pyridin-2-yl group, pyridin-3-yl group, pyridin-4-yl group, thiophen-2-yl group, pyrrol-2-yl group, furan-2-yl group, hydroxyphenyl group, or a C₁-C₆ alkyl group.

In another particular embodiment of the present disclosure, Z represents pyridyl group, thienyl group, pyrrolyl group, furyl group, hydroxyphenyl group, or methyl group.

In a more particular embodiment of the present disclosure, Z represents pyridin-2-yl group, thiophen-2-yl group, pyrrol-2-yl group, furan-2-yl group, phenol group, or methyl group, preferably Z represents pyridin-2-yl group.

In a particular embodiment of the present disclosure, R₁ represents hydrogen, a C₁-C₆ alkyl group, a C₃-C₆ cycloalkyl group, or phenyl group.

In another particular embodiment of the present disclosure, R₁ represents hydrogen, methyl group, ethyl group, tert-butyl group, cyclopropyl group, phenyl group, or pyridyl group, preferably R₁ represents hydrogen, methyl group, ethyl group, phenyl group, or pyridyl group.

In a particular embodiment of the present disclosure, X represents O or S, preferably X represents S.

In a particular embodiment of the present disclosure,

R₂ or R₃ represents hydrogen, a C₁-C₆ alkyl group, a C₂-C₆ alkenyl group, a C₃-C₆ cycloalkyl group, —NR₅R₆, pyridyl group, furyl group, phenyl group, a (C₁-C₆ alkyl)-pyridyl group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OH group; or

R₂ and R₃ together with the nitrogen atom to which they are attached form a 4- to 6-membered heterocycloalkyl;
in which R₇ is a —O—(C₁-C₆ alkyl) group or a —NH—N═CR₈-pyridyl group;
in which R₈ is a C₁-C₆ alkyl group.

In a more particular embodiment of the present disclosure,
R₂ and R₃ represents hydrogen, or a C₁-C₆ alkyl group; or
either of R₂ and R₃ represents H, the other of R₂ and R₃ represents a C₁-C₆ alkyl group, a C₂-C₆ alkenyl group, a C₃-C₆ cycloalkyl group, —NR₅R₆, pyridyl group, furyl group, phenyl group, a (C₁-C₆ alkyl)-pyridyl group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OH group; or
R₂ and R₃ together with the nitrogen atom to which they are attached form a 4- to 6-membered heterocycloalkyl;
   in which R₇ is a —O-(C₁-C₆ alkyl) group or a —NH—N═CR₈-pyridyl group;
      in which R₈ is a C₁-C₆ alkyl group.

In an especially more particular embodiment of the present disclosure,
R₂ and R₃ represents hydrogen, or a C₁-C₆ alkyl group; or
either of R₂ and R₃ represents H, the other of R₂ and R₃ represents a C₁-C₆ alkyl group, a C₂-C₆ alkenyl group, a C₃-C₆ cycloalkyl group, —NR₅R₆, pyridin-2-yl group, pyridin-3-yl group, furan-2-yl group, phenyl group, a (C₁-C₆ alkyl)-(pyridin-2-yl) group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OH group; or
R₂ and R₃ together with the nitrogen atom to which they are attached form a 4- to 6-membered heterocycloalkyl;
   in which R₇ is a —O-(C₁-C₆ alkyl) group or a —NH—N═CR₈-(pyridin-2-yl) group;
      in which R₈ is a C₁-C₆ alkyl group.

In another particular embodiment of the present disclosure,

R₂ and R₃ represent hydrogen, methyl group, or ethyl group; or

either of R₂ and R₃ represents H, the other of R₂ and R₃ represents methyl group, propyl group, —NH₂, —CH₂CH═CH₂, —CH₂CH₂N(CH₃)₂, —CH₂CH₂OH, —CH₂COOCH₂CH₃, —CH₂CONH—N═C(CH₃)-pyridyl group, —CH₂-pyridyl group, pyridyl group, furyl group, phenyl group, benzyl group, cyclopropyl group, or cyclohexyl group; or

R₂ and R₃ together with the nitrogen atom to which they are attached form an azetidinyl group or a piperidinyl group.

In a more particular embodiment of the present disclosure,

either of R₂ and R₃ represents H, the other of R₂ and R₃ represents methyl group, propyl group, —NH₂, —CH₂CH═CH₂, —CH₂CH₂N(CH₃)₂, —CH₂CH₂OH, —CH₂COOCH₂CH₃, —CH₂CONH—N═C(CH₃)-(pyridin-2-yl) group, —CH₂-(pyridin-2-yl) group, pyridin-2-yl group, pyridin-3-yl group, furan-2-yl group, phenyl group, benzyl group, cyclopropyl group, or cyclohexyl group.

In a particular embodiment of the present disclosure,

when Z represents pyridyl group (particularly pyridine-2-yl) and X represents S,

R₁ does not represent methyl, and

R₂ and R₃ together with the nitrogen atom to which they are attached do not form an azetidinyl group.

In a particular embodiment of the present disclosure,

when Z represents pyridyl group (particularly pyridine-2-yl) and X represents S,

R₁ does not represent pyridyl group (particularly pyridine-2-yl), and

R₂ and R₃ do not represent methyl.

Especially preferred embodiments of the present disclosure are compounds of the general formula in which
Z represents pyridyl group, preferably pyridin-2-yl group;
X represents S;
R₁ represents C₁-C₆ alkyl group, preferably methyl;
either of R₂ and R₃ represents H, the other of R₂ and R₃ represents —NH₂;
   and the diastereomers, enantiomers, metabolites, salts, solvates thereof or solvates of the salts thereof.

Very particular preference is given to the following compounds according to the disclosure:
(E)-N′-(1-(pyridin-2-yl)ethylidene)azetidine-1-carbohydrazide,
(E)-N′-(2,2-dimethyl-1-(pyridin-2-yl)propylidene)azetidine-1-carbothiohydrazide,
(E)-N′-(pyridin-2-ylmethylene)azetidine-1-carbothiohydrazide,
(E)-N′-(1-(pyridin-2-yl)propylidene)azetidine-1-carbothiohydrazide,
(E)-N′-(1-(pyridin-3-yl)ethylidene)azetidine-1-carbothiohydrazide,
(E)-N′-(1-(pyridin-4-yl)ethylidene)azetidine-1-carbothiohydrazide,
(E)-N′-(phenyl (pyridin-2-yl)methylene)azetidine-1-carbothiohydrazide,
(E)-N′-(1-(1H-pyrrol-2-yl)ethylidene)azetidine-1-carbothiohydrazide,
N′-(propan-2-ylidene)azetidine-1-carbothiohydrazide,
(E)-N′-(1-(2-hydroxyphenyl)ethylidene)azetidine-1-carbothiohydrazide,
(E)-2-(1-(pyridin-2-yl)ethylidene)hydrazine-1-carbothioamide,
(E)-N,N-dimethyl-2-(1-(pyridin-2-yl)ethylidene)hydrazine-1-carbothioamide,
(E)-N,N-diethyl-2-(1-(pyridin-2-yl)ethylidene)hydrazine-1-carbothioamide,
(E)-N′-(1-(pyridin-2-yl)ethylidene)piperidine-1-carbothiohydrazide,
(E)-N-phenyl-2-(1-(pyridin-2-yl)ethylidene)hydrazine-1-carbothioamide,
(E)-N-(pyridin-2-yl)-2-(1-(pyridin-2-yl)ethylidene)hydrazine-1-carbothioamide,
(E)-N-propyl-2-(1-(pyridin-2-yl)ethylidene)hydrazine-1-carbothioamide,
(E)-N-benzyl-2-(1-(pyridin-2-yl)ethylidene)hydrazine-1-carbothioamide,
(E)-N-cyclohexyl-2-(1-(pyridin-2-yl)ethylidene)hydrazine-1-carbothioamide,
(E)-N′-(1-(pyridin-2-yl)ethylidene)hydrazinecarbothiohydrazide,
2-(propan-2-ylidene)hydrazine-1-carbothioamide,
(E)-2-(1-(thiophen-2-yl)ethylidene)hydrazine-1-carbothioamide,
(E)-2-(1-(pyridin-2-yl)propylidene)hydrazine-1-carbothioamide,
(E)-2-(1-(furan-2-yl)ethylidene)hydrazine-1-carbothioamide,
(E)-2-(cyclopropyl (pyridin-2-yl)methylene)hydrazine-1-carbothioamide,
(E)-N′-(1-(thiophen-2-yl)ethylidene)hydrazinecarbothiohydrazide,
(E)-N′-(1-(furan-2-yl)ethylidene)hydrazinecarbothiohydrazide,
(E)-N-(pyridin-2-yl)-2-(1-(thiophen-2-yl)ethylidene)hydrazine-1-carbothioamide,
(E)-2-(1-(furan-2-yl)ethylidene)-N-(pyridin-2-yl)hydrazine-1-carbothioamide,
(E)-N-methyl-2-(1-(pyridin-2-yl)ethylidene)hydrazine-1-carbothioamide,
(E)-N-allyl-2-(1-(pyridin-2-yl)ethylidene)hydrazine-1-carbothioamide,
(E)-N-cyclopropyl-2-(1-(pyridin-2-yl)ethylidene)hydrazine-1-carbothioamide,
(E)-N-(2-(dimethylamino)ethyl)-2-(1-(pyridin-2-yl)ethylidene)hydrazine-1-carbothioamide,
(E)-N-(furan-2-ylmethyl)-2-(1-(pyridin-2-yl)ethylidene)hydrazine-1-carbothioamide,
(E)-2-(1-(pyridin-2-yl)ethylidene)-N-(pyridin-2-ylmethyl)hydrazine-1-carbothioamide,
(E)-2-(1-(pyridin-2-yl)ethylidene)-N-(pyridin-3-yl)hydrazine-1-carbothioamide,
(E)-N-(2-hydroxyethyl)-2-(1-(pyridin-2-yl)ethylidene)hydrazine-1-carbothioamide,
Ethyl (E)-(2-(1-(pyridin-2-yl)ethylidene)hydrazine-1-carbonothioyl)glycinate, and
(E)-N-(2-oxo-2-(2-((E)-1-(pyridin-2-yl)ethylidene)hydrazinyl)ethyl)-2-(1-(pyridin-2-yl)ethylidene)hydrazine-1-carbothioamide.

Especially particular preference is given to the following compounds according to the disclosure:
(E)-N′-(pyridin-2-ylmethylene)azetidine-1-carbothiohydrazide,
(E)-N′-(1-(pyridin-2-yl)propylidene)azetidine-1-carbothiohydrazide,
(E)-N′-(phenyl (pyridin-2-yl)methylene)azetidine-1-carbothiohydrazide,
(E)-2-(1-(pyridin-2-yl)ethylidene)hydrazine-1-carbothioamide,
(E)-N,N-dimethyl-2-(1-(pyridin-2-yl)ethylidene)hydrazine-1-carbothioamide,
(E)-N,N-diethyl-2-(1-(pyridin-2-yl)ethylidene)hydrazine-1-carbothioamide,
(E)-N′-(1-(pyridin-2-yl)ethylidene)piperidine-1-carbothiohydrazide,
(E)-N-phenyl-2-(1-(pyridin-2-yl)ethylidene)hydrazine-1-carbothioamide,
(E)-N-(pyridin-2-yl)-2-(1-(pyridin-2-yl)ethylidene)hydrazine-1-carbothioamide,
(E)-N-propyl-2-(1-(pyridin-2-yl)ethylidene)hydrazine-1-carbothioamide,
(E)-N-benzyl-2-(1-(pyridin-2-yl)ethylidene)hydrazine-1-carbothioamide,
(E)-N-cyclohexyl-2-(1-(pyridin-2-yl)ethylidene)hydrazine-1-carbothioamide,
(E)-N′-(1-(pyridin-2-yl)ethylidene)hydrazinecarbothiohydrazide,
(E)-N-methyl-2-(1-(pyridin-2-yl)ethylidene)hydrazine-1-carbothioamide,
(E)-N-allyl-2-(1-(pyridin-2-yl)ethylidene)hydrazine-1-carbothioamide,
(E)-N-cyclopropyl-2-(1-(pyridin-2-yl)ethylidene)hydrazine-1-carbothioamide,
(E)-N-(2-(dimethylamino)ethyl)-2-(1-(pyridin-2-yl)ethylidene)hydrazine-1-carbothioamide,
(E)-N-(furan-2-ylmethyl)-2-(1-(pyridin-2-yl)ethylidene)hydrazine-1-carbothioamide,
(E)-2-(1-(pyridin-2-yl)ethylidene)-N-(pyridin-2-ylmethyl)hydrazine-1-carbothioamide,
(E)-N-(2-hydroxyethyl)-2-(1-(pyridin-2-yl)ethylidene)hydrazine-1-carbothioamide,
Ethyl (E)-(2-(1-(pyridin-2-yl)ethylidene)hydrazine-1-carbonothioyl)glycinate, and
(E)-N-(2-oxo-2-(2-((E)-1-(pyridin-2-yl)ethylidene)hydrazinyl)ethyl)-2-(1-(pyridin-2-yl)ethylidene)hydrazine-1-carbothioamide.

Especially more preferred compound according to the present disclosure is (E)-N′-(1-(pyridin-2-yl)ethylidene)hydrazinecarbothiohydrazide.

The compounds according to the disclosure act as chemical activators of NMNAT2 and have an unforeseeable useful pharmacological activity spectrum.

Thus, the present disclosure also provides a method for treating and/or preventing diseases (especially neurodegeneration and age-associated diseases or conditions associated with NAD loss) in human and animals, comprising administering at least one compound according to the present disclosure.

Further, the present disclosure also provides the use of the compounds according to the disclosure in the manufacture of a medicament for treatment and/or prevention of diseases (especially neurodegeneration and age-associated diseases or conditions associated with NAD loss) in human and animals.

Treatment of and/or prevention of amyotrophic lateral sclerosis, Parkinson's disease, traumatic brain injury, neonatal nerve crush injury, Alzheimer's disease, Chemotherapy-induced peripheral neuropathy (CIPN), ischemia, retinal degeneration, age-associated deficiency of neurogenesis, hypoadiponectinemia, and multi-organ insulin resistance is particularly preferred.

The present disclosure further also provides a method for treatment and/or prevention of diseases, especially the diseases mentioned above, using an effective amount of at least one of the compounds according to the disclosure.

In the context of the present disclosure, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prophylaxis”, “prevention” and “preclusion” are used synonymously in the context of the present disclosure and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

The compounds according to the disclosure can be used alone or, if required, in combination with other active ingredients. The present disclosure further provides medicaments comprising at least one of the compounds according to the disclosure and one or more further active ingredients, in particular for the treatment and/or prevention of the diseases mentioned above.

The compounds according to the disclosure can act systemically and/or locally. For this purpose, they can be administered in a suitable manner, for example by the oral, parenteral, pulmonal, nasal, sublingual, lingual, buccal, rectal, dermal, transdermal or conjunctival route, via the ear or as an implant or stent.

The compounds according to the disclosure can be administered in administration forms suitable for these administration routes.

Suitable administration forms for oral administration are those which work according to the prior art and release the compounds according to the disclosure rapidly and/or in a modified manner and which contain the compounds according to the disclosure in crystalline and/or amorphous and/or dissolved form, for example tablets (uncoated or coated tablets, for example with gastric juice-resistant or retarded-dissolution or insoluble coatings which control the release of the inventive compound), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalation medicaments (including powder inhalers, nebulizers), nasal drops, solutions or sprays; tablets for lingual, sublingual or buccal administration, films/oblates or capsules, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Preference is given to oral or parenteral administration.

The compounds according to the disclosure can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctors.

The present disclosure further provides a pharmaceutical preparation which comprises at least one compound according to the disclosure, typically together with one or more inert, nontoxic, pharmaceutically suitable excipients, and the use thereof for the aforementioned purposes.

The present disclosure provides a method for high throughput screening of NMNAT2 activators, comprising
(a) adding compounds to NMNAT2 enzyme reaction mixture;
(b) initiating NMNAT2 enzyme reaction by adding NMN;
(c) monitoring the conversion of NMN to NADH by measuring fluorescence intensity of NADH at an excitation of 340 nm and an emission of 445 nm (Y axis) as a function of time (X axis), and then calculating reaction rate;
(d) obtaining relative NMNAT2 enzyme reaction rate in the presence of each compound by normalizing with DMSO-treated control; and
(e) selecting compounds with the relative reaction rate higher than 120% as NMNAT2 activators.

In a particular embodiment of the method for high throughput screening of NMNAT2 activators, the method further comprises
(f) selecting and re-testing the compounds with the relative reaction rate higher than 120% for ADH enzyme assay;
(g) adding the selected compounds to ADH enzyme reaction mixture;
(h) initiating ADH enzyme reaction by adding NAD;
(i) monitoring the conversion of NAD to NADH by measuring fluorescence intensity of NADH at an excitation of 340 nm and an emission of 445 nm (Y axis) as a function of time (X axis), and calculating reaction rate;
(j) obtaining relative ADH enzyme reaction rate in the presence of each compound by normalizing with DMSO-treated control; and
(k) selecting compounds that are active (relative reaction rate higher than 120%) in NMNAT2 enzyme assay but inactive (relative reaction rate lower than or equivalent to 100%) in the ADH enzyme assay as NMNAT2 activators.

In a particular embodiment of the present method, wherein in step (a), the NMNAT2 enzyme reaction mixture contains or consists of Tris-Cl, MgCl₂, ethanol, ATP, semicarbizide, bovine serum albumin, NMNAT2, and ADH; preferably 50 mM Tris-Cl (pH 8.0), 12 mM MgCl₂, 1.5% ethanol, 2.5 mM ATP, 10 mM semicarbizide, 0.2% bovine serum albumin, 0.1 μg/ml NMNAT2, and 0.4 units ADH.

In a particular embodiment of the present method, wherein in step (a) or (g), the compound is added at the same final concentration or different final concentration, for example, at a concentration ranging from 3-8 μM (such as at a concentration of 5 μM), or at a series of concentration ranging from 1-100 μM (such as at a series of concentration of 1, 3, 10, 30 and 100 μM).

In a particular embodiment of the present method, wherein in step (b), NMN is added at a concentration of 30-70 μM, preferably 50 μM.

In a particular embodiment of the present method, wherein the step (b) is followed by gentle mixing.

In a particular embodiment of the present method, wherein in step (c) or (i), monitoring is performed for 20-40 minutes (such as 30 minutes) at room temperature.

In a particular embodiment of the present method, wherein in step (c) or (i), the reaction rate is calculated by using the equation: reaction rate=Δ F_{340/445}/Δt.

In a particular embodiment of the present method, wherein in step (e) and/or (f), the selected compounds have relative reaction rate higher than 120%.

In a particular embodiment of the present method, wherein in step (g), the ADH enzyme reaction mixture contains or consists of Tris-Cl, MgCl₂, ethanol, semicarbizide, bovine serum albumin, and ADH; preferably 50 mM Tris-Cl (pH 8.0), 12 mM MgCl₂, 1.5% ethanol, 10 mM semicarbizide, 0.2% bovine serum albumin, and 0.04 units ADH.

In a particular embodiment of the present method, wherein in step (h), NAD is added at a concentration of 30-60 μM, preferably 50 μM.

In a particular embodiment of the present method, wherein the step (h) is followed by gentle mixing.

### Preparation of the Compounds According to the Disclosure

The preparation of the compounds according to the disclosure is illustrated by the synthesis schemes which follow.

### Synthesis of the Pyridine Thiosemicarbazones (Including Compounds NSC and 2-39)

General Method A: A general synthetic approach to the preparation of thiosemicarbazones is shown in Scheme 1.

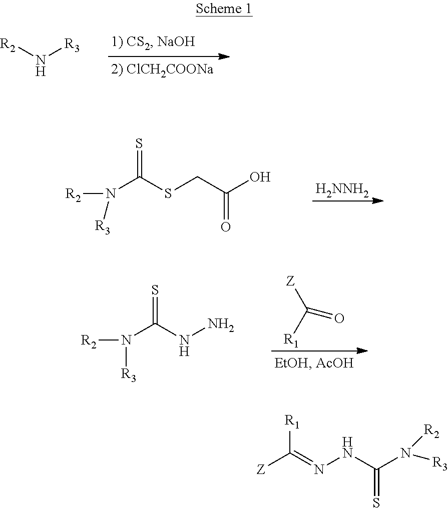

in which groups Z, R₁, R₂, and R₃ are defined above.

General Method B: Another general synthetic approach to the preparation of thiosemicarbazones is shown in Scheme 2.

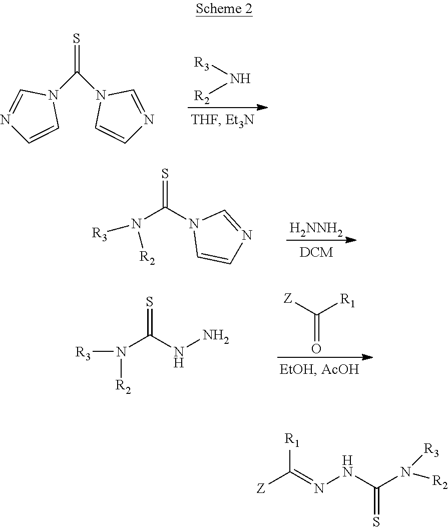

in which groups Z, R₁, R₂, and R₃ are defined above.

General Method C: Another general synthetic approach to the preparation of thiosemicarbazones is shown in Scheme 3.

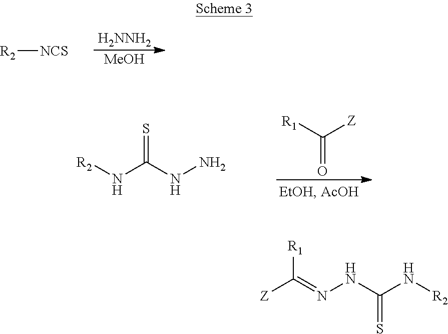

in which groups Z, R₁, and R₂ are defined above.

General Method D: Another general synthetic approach to the preparation of thiosemicarbazones is shown in Scheme 4.

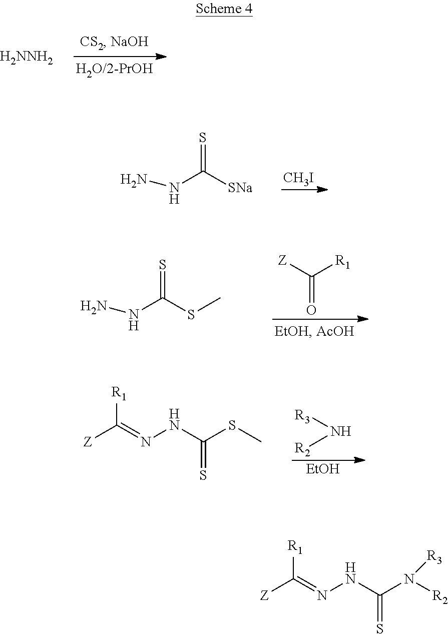

in which groups Z, R₁, R₂, and R₃ are defined above.

### Synthesis of the Pyridine Semicarbazones (Including Compound 1)

Another general synthetic approach to the preparation of semicarbazones is shown in Scheme 5.

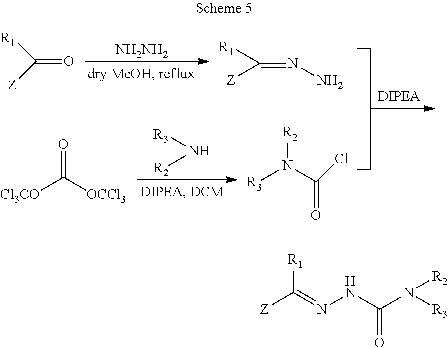

in which groups Z, R₁, R₂, and R₃ are defined above.

The working examples which follow illustrate the disclosure. The disclosure is not restricted to the examples.

Unless stated otherwise, the percentages in the tests and examples which follow are percentages by weight; parts are parts by weight. Solvent ratios, dilution ratios and concentration data for liquid/liquid solutions are based in each case on volume.

### PREPARATION EXAMPLES

### Example 1

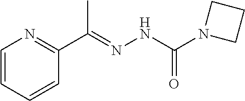 [SMILES: C/C(=N\NC(=O)N1CCC1)c1ccccn1]

(E)-N′-(1-(pyridin-2-yl)ethylidene)azetidine-1-carbohydrazide (Compound 1)

Azetidine (85 mg, 1.5 mmol) was dissolved in 5 mL methylene chloride and diisopropylethylamine (195 mg, 1.50 mmol) was added followed by triphosgene (267 mg, 0.9 mmol) and the reaction mixture were stirred at room temperature for 4 h to give the azetidine-1-carbonyl chloride in situ. Without workup, (E)-2-(1-hydrazonoethyl)pyridine (203 mg, 1.5 mmol) was added directly to this mixture followed by additional diisopropylethylamine (195 mg, 1.50 mmol) and the reaction was stirred at room temperature for 72 h. The reaction was then evaporated to dryness and purified by silica gel chromatography to afford compound 1 as a white crystalline solid. ¹H NMR (400 MHZ, CDCl₃) δ 8.58 (d, J=4.8 Hz, 1H), 7.98 (s, 1H), 7.92 (d, J=8.1 Hz, 1H), 7.68 (td, J=7.8, 1.9 Hz, 1H), 7.24 (dd, J=7.4, 4.9 Hz, 1H), 4.36 (t, J=7.8 Hz, 4H), 2.40-2.27 (m, 5H). ¹³C NMR (101 MHZ, CDCl₃) δ 156.12, 155.55, 148.55, 146.01, 136.10, 123.18, 119.89, 16.96, 10.05.

### Example 2

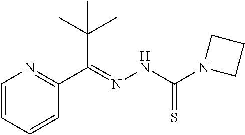 [SMILES: CC(C)(C)/C(=N\NC(=S)N1CCC1)c1ccccn1]

(E)-N′-(2,2-dimethyl-1-(pyridin-2-yl)propylidene)azetidine-1-carbothiohydrazide (Compound 2)

General Method A: Carbon disulfide (0.02 mol) was added dropwise to azetidine (0.02 mol) in NaOH solution (25 mL, 0.8 M) and this mixture was allowed to react overnight at room temperature. Next, sodium chloroacetate (0.02 mol) was added to the aqueous and allowed to react for 24 h at room temperature. The addition of concentrated HCl (2.5 mL) gave the solid carboxymethyl thiocarbamate intermediate. Approximately 8 mmol of carboxymethyl thiocarbamate was dissolved in 2 mL of hydrazine hydrate plus 1 mL of water. This was followed by five cycles of gentle heating (until fuming) and cooling. The solution was then allowed to stand until fine white crystal of thiosemicarbazide intermediate was formed. 1.0 mmol of this intermediate was added to 2,2-dimethyl-1-(pyridin-2-yl)propan-1-one (1.0 mmol) dissolved in EtOH (1.5 mL). Then, a drop of glacial acetic acid were added and the mixture was refluxed for 12 h and cooled to 5° C. to afford compound 2 as a crystalline white solid (140 mg, 51%). ¹H NMR (400 MHZ, CDCl₃) δ 8.78 (ddd, J=4.9, 1.8, 1.0 Hz, 1H), 8.27 (s, 1H), 7.83 (td, J=7.7, 1.8 Hz, 1H), 7.37 (ddd, J=7.7, 4.9, 1.2 Hz, 1H), 7.20 (dt, J=7.8, 1.1 Hz, 1H), 4.68 (t, J=7.8 Hz, 2H), 4.28 (t, J=7.9 Hz, 2H), 2.40-2.27 (m, 2H), 1.20 (s, 9H). ¹³C NMR (101 MHZ, CDCl₃) δ 174.99, 157.69, 151.70, 150.87, 136.97, 123.88, 123.68, 56.76, 53.47, 28.66, 16.70.

### Example 3

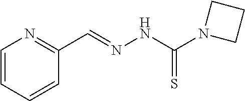 [SMILES: S=C(N/N=C/c1ccccn1)N1CCC1]

(E)-N′-(pyridin-2-ylmethylene)azetidine-1-carbothiohydrazide (Compound 3)

Following General Method A, compound 3 was isolated as a white solid. Yield 47%. ¹H NMR (400 MHZ, DMSO-d₆) δ 11.70 (s, 1H), 8.57 (dt, J=4.8, 1.4 Hz, 1H), 8.05 (d, J=0.9 Hz, 1H), 7.86-7.79 (m, 2H), 7.37 (td, J=4.9, 3.5 Hz, 1H), 4.61 (s, 2H), 4.11 (s, 2H), 2.33-2.21 (m, 2H). ¹³C NMR (101 MHz, DMSO-d₆) δ 175.37, 153.80, 149.93, 143.06, 137.27, 124.36, 119.79, 57.07, 53.50, 16.49. MS: 221.0858 [M+H]⁺.

### Example 4

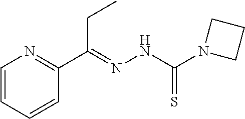 [SMILES: CC/C(=N\NC(=S)N1CCC1)c1ccccn1]

(E)-N′-(1-(pyridin-2-yl)propylidene)azetidine-1-carbothiohydrazide (Compound 4)

Following General Method A, compound 4 was isolated as a tawny solid. Yield 41%. ¹H NMR (400 MHz, CDCl₃) δ 8.87 (s, 1H), 8.61 (d, J=4.3 Hz, 1H), 7.85 (d, J=8.0 Hz, 1H), 7.69 (td, J=7.7, 1.8 Hz, 1H), 7.29-7.22 (m, 1H), 4.73 (s, 2H), 4.36 (s, 2H), 2.93 (q, J=7.7 Hz, 2H), 2.38 (dq, J=14.9, 7.5 Hz, 3H), 1.18 (t, J=7.7 Hz, 3H). MS: 249.1177 [M+H]⁺.

### Example 5

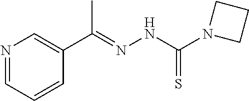 [SMILES: C/C(=N\NC(=S)N1CCC1)c1cccnc1]

(E)-N′-(1-(pyridin-3-yl)ethylidene)azetidine-1-carbothiohydrazide (Compound 5)

Following General Method A, compound 5 was isolated as a white solid. Yield 55%. ¹H NMR (400 MHz, CDCl₃) δ 8.94 (dd, J=2.4, 0.9 Hz, 1H), 8.69 (s, 1H), 8.62 (dd, J=4.8, 1.6 Hz, 1H), 7.93 (ddd, J=8.0, 2.4, 1.6 Hz, 1H), 7.34 (ddd, v8.1, 4.8, 0.9 Hz, 1H), 4.70 (t, J=7.9 Hz, 2H), 4.36 (t, J=7.8 Hz, 2H), 2.44-2.33 (m, 2H), 2.24 (s, 3H). ¹³C NMR (101 MHz, CDCl₃) δ 175.20, 150.01, 147.54, 143.29, 133.44, 133.06, 123.29, 56.78, 53.73, 16.73, 12.03. MS: 235.1020 [M+H]⁺.

### Example 6

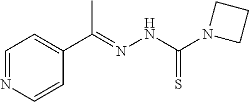 [SMILES: C/C(=N\NC(=S)N1CCC1)c1ccncc1]

(E)-N′-(1-(pyridin-4-yl)ethylidene)azetidine-1-carbothiohydrazide (Compound 6)

Following General Method A, compound 6 was isolated as a white solid. Yield 52%. ¹H NMR (400 MHz, CDCl₃) δ 8.74 (s, 1H), 8.69-8.64 (m, 2H), 7.56-7.50 (m, 2H), 4.71 (t, J=7.8 Hz, 2H), 4.36 (t, J=7.9 Hz, 2H), 2.46-2.36 (m, 2H), 2.22 (s, 3H). ¹³C NMR (101 MHz, CDCl₃) δ 175.27, 150.32, 144.92, 143.22, 119.94, 56.87, 53.82, 16.77, 11.64. MS: 235.1020 [M+H]⁺.

### Example 7

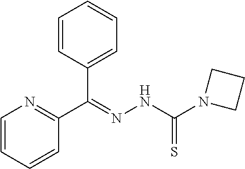 [SMILES: S=C(N/N=C(\c1ccccc1)c1ccccn1)N1CCC1]

(E)-N′-(phenyl(pyridin-2-yl)methylene)azetidine-1-carbothiohydrazide (Compound 7)

Following General Method A, compound 7 was isolated as a yellow solid. Yield 42%. ¹H NMR (400 MHZ, CDCl₃) δ 13.42 (s, 1H), 8.84 (ddd, J=4.9, 1.9, 0.9 Hz, 1H), 7.79 (td, J=7.8, 1.8 Hz, 1H), 7.52-7.46 (m, 2H), 7.45-7.40 (m, 3H), 7.30 (dt, J=8.2, 1.1 Hz, 1H), 4.69 (t, J=7.8 Hz, 2H), 4.35 (t, J=7.9 Hz, 2H), 2.39-2.27 (m, 2H). ¹³C NMR (101 MHZ, CDCl₃) δ 175.52, 152.30, 148.69, 137.76, 137.13, 129.63, 128.86, 128.75, 128.40, 125.73, 123.92, 56.92, 53.44, 16.80. MS: 297.1715 [M+H]⁺.

### Example 8

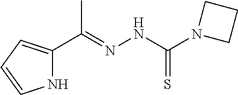

(E)-N′-(1-(1H-pyrrol-2-yl)ethylidene)azetidine-1-carbothiohydrazide (Compound 8)

Following General Method A, compound 8 was isolated as a yellow solid. Yield 49%. ¹H NMR (400 MHZ, DMSO-d₆) δ 10.74 (s, 1H), 9.87 (s, 1H), 6.85 (td, J=2.7, 1.5 Hz, 1H), 6.51 (td, J=3.2, 2.6, 1.6 Hz, 1H), 6.12-6.06 (m, 1H), 4.69-3.97 (m, 4H), 2.25-2.13 (m, 5H). ¹³C NMR (101 MHZ, DMSO-d₆) δ 176.32, 144.62, 130.28, 122.13, 111.51, 109.17, 16.33, 13.98. MS: 223.1016 [M+H]⁺.

### Example 9

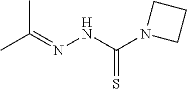

N′-(propan-2-ylidene)azetidine-1-carbothiohydrazide (Compound 9)

Following General Method A, compound 9 was isolated as a white solid. Yield 21%. ¹H NMR (400 MHz, CDCl₃) δ 8.29 (s, 1H), 4.42 (d, J=93.6 Hz, 4H), 2.34-2.25 (m, 2H), 1.96 (s, 3H), 1.83 (s, 3H). ¹³C NMR (101 MHZ, CDCl₃) δ 175.32, 147.79, 56.23, 53.28, 16.65, 15.48. MS: 172.091 [M+H]⁺.

### Example 10

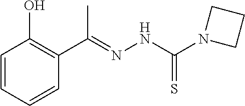 [SMILES: C/C(=N\NC(=S)N1CCC1)c1ccccc1O]

(E)-N′-(1-(2-hydroxyphenyl)ethylidene)azetidine-1-carbothiohydrazide (Compound 10)

Following General Method A, compound 10 was isolated as a yellow solid. Yield 40%. ¹H NMR (400 MHZ, DMSO-d₆) δ 12.55 (s, 1H), 10.15 (s, 1H), 7.57 (d, J=7.9 Hz, 1H), 7.27 (t, J=7.7 Hz, 1H), 6.88 (t, J=7.5 Hz, 2H), 4.21 (t, J=7.5 Hz, 4H), 2.37 (s, 3H), 2.28 (p, J=7.7 Hz, 2H). ¹³C NMR (101 MHz, DMSO-d₆) δ 179.37, 158.22, 155.35, 131.18, 128.72, 120.56, 118.93, 117.59, 53.16, 15.50, 14.64. MS: 250.1016 [M+H]⁺.

### Example 11

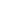

(E)-2-(1-(pyridin-2-yl)ethylidene)hydrazine-1-carbothioamide (Compound 11)

To a solution of 2-acetylpyridine (1.0 mmol) and thiosemicarbazide (1.0 mmol) in EtOH (1 ml) was added AcOH (1 drop). After stirring at reflux overnight, the solution was cooled to 5° C. and compound 11 was isolated as a yellow solid (116 mg, 60%). ¹H NMR (400 MHZ, CDCl₃) δ 8.92 (s, 1H), 8.66-8.57 (m, 1H), 7.98 (d, J=7.9 Hz, 1H), 7.72 (td, J=7.8, 1.7 Hz, 1H), 7.42 (s, 1H), 7.34-7.29 (m, 1H), 6.82-6.70 (m, 1H), 2.45 (s, 3H). ¹³C NMR (101 MHZ, CDCl₃) δ 179.32, 154.28, 149.13, 148.93, 136.39, 124.28, 120.45, 11.65.

### Example 12

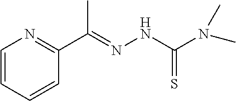

(E)-N,N-dimethyl-2-(1-(pyridin-2-yl)ethylidene)hydrazine-1-carbothio amide (Compound 12)

Following General Method A, compound 12 was isolated as a yellow solid. Yield 41%.

### Example 13

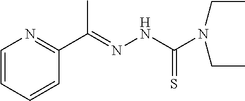 [SMILES: CCN(CC)C(=S)N/N=C(\C)c1ccccn1]

(E)-N,N-diethyl-2-(1-(pyridin-2-yl)ethylidene)hydrazine-1-carbothioamide (Compound 13)

Following General Method A, compound 13 was isolated as a yellow solid. Yield 41%. ¹H NMR (400 MHZ, CDCl₃) δ 9.96 (s, 1H), 8.59 (ddd, J=4.9, 1.8, 0.9 Hz, 1H), 8.07 (dt, J=8.2, 1.1 Hz, 1H), 7.70 (td, J=7.7, 1.8 Hz, 1H), 7.23 (ddd, J=7.4, 4.8, 1.2 Hz, 1H), 3.48 (q, J=7.1 Hz, 4H), 2.52 (d, J=1.8 Hz, 3H), 1.27 (t, J=7.1 Hz, 6H). ¹³C NMR (101 MHZ, CDCl₃) δ 163.52, 148.46, 146.93, 135.97, 122.88, 120.10, 46.14, 12.76, 11.70.

### Example 14

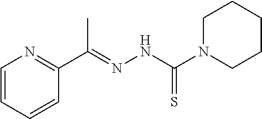 [SMILES: C/C(=N\NC(=S)N1CCCCC1)c1ccccn1]

(E)-N′-(1-(pyridin-2-yl)ethylidene)piperidine-1-carbothiohydrazide (Compound 14)

Following General Method A, compound 14 was isolated as a yellow solid. Yield 45%.

### Example 15

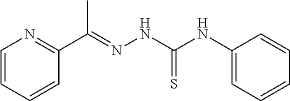 [SMILES: C/C(=N\NC(=S)Nc1ccccc1)c1ccccn1]

(E)-N-phenyl-2-(1-(pyridin-2-yl)ethylidene)hydrazine-1-carbothioamide (Compound 15)

Following General Method A, compound 15 was isolated as a white solid. Yield 40%. ¹H NMR (400 MHZ, CDCl₃) δ 9.41 (s, 1H), 8.89 (s, 1H), 8.66 (ddd, J=4.8, 1.8, 0.9 Hz, 1H), 8.04 (dt, J=8.1,1.1 Hz, 1H), 7.77 (ddd, J=8.1, 7.5, 1.8 Hz, 1H), 7.74-7.70 (m, 2H), 7.47-7.41 (m, 2H), 7.34 (ddd, J=7.5, 4.9, 1.2 Hz, 1H), 7.31-7.26 (m, 1H), 2.50 (s, 3H). ¹³C NMR (101 MHz, CDCl₃) δ 149.02, 148.09, 137.78, 136.42, 128.86, 126.30, 124.28, 124.22, 120.35, 11.68.

### Example 16

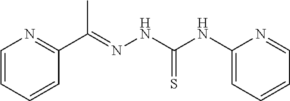 [SMILES: C/C(=N\NC(=S)Nc1ccccn1)c1ccccn1]

(E)-N-(pyridin-2-yl)-2-(1-(pyridin-2-yl)ethylidene)hydrazine-1-carbothioamide (Compound 16)

General Method B: A mixture of 2-aminopyridine (10 mmol), TEA (10 mmol) and 1,1′-thiocarbonyldiimidazole (10 mmol) in THF (20 mL) was stirred at room temperature overnight. The reaction mixture was filtered and the filtrate was concentrated under reduced pressure before addition of hydrazine (30 mmol). The resulting reaction mixture was stirred at room temperature for about 3 h and then partitioned between DCM and water. The organic layer was dried over MgSO₄, filtered, and concentrated under reduced pressure to give the thiosemicarbazide intermediate. To a solution of 2-acetylpyridine (1.0 mmol) and thiosemicarbazide intermediate (1.0 mmol) in EtOH (1 ml) was added AcOH (1 drop). After stirring at reflux overnight, the solution was concentrated and purified via chromatography on silica gel to afford compound 16 as a yellow solid (116 mg, 60%). ¹H NMR (400 MHZ, DMSO-d₆) δ 15.01 (s, 1H), 11.14 (s, 1H), 8.64 (d, J=4.8 Hz, 1H), 8.38 (d, J=5.0 Hz, 1H), 8.19 (d, J=8.0 Hz, 1H), 7.94-7.83 (m, 2H), 7.44 (dd, J=7.4, 4.9 Hz, 1H), 7.30 (d, J=8.3 Hz, 1H), 7.13 (t, J=6.1 Hz, 1H), 2.51 (s, 3H). ¹³C NMR (101 MHz, DMSO-d₆) δ 178.42, 155.11, 153.96, 149.15, 146.24, 139.98, 137.10, 124.69, 121.22, 118.61, 113.39, 99.98, 13.31.

### Example 17

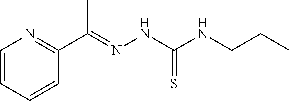 [SMILES: CCCNC(=S)N/N=C(\C)c1ccccn1]

(E)-N-propyl-2-(1-(pyridin-2-yl)ethylidene)hydrazine-1-carbothioamide (Compound 17)

General Method C: To a solution of propyl isothiocyanate (10 mmol) in MeOH (20 ml) was added hydrazine hydrate (10 mmol) at 0° C. After stirring for about 1 h, the solution was concentrated under reduced pressure and recrystallization from MeOH to afford the thiosemicarbazide intermediate. A mixture of 2-acetylpyridine (1.0 mmol), thiosemicarbazide intermediate (1.0 mmol) and AcOH (1 drop) in EtOH (1 ml) was stirred at reflux overnight. After the reaction finished, the solution was cooled to 5° C. to afford compound 17 as a yellow solid (136 mg, 58%). ¹H NMR (400 MHz, CDCl₃) δ 14.52 (s, 1H), 8.77 (ddd, J=4.9, 1.9, 0.9 Hz, 1H), 7.87 (td, J=7.9, 1.9 Hz, 1H), 7.79 (s, 1H), 7.57-7.53 (m, 1H), 7.37 (ddd, J=7.7, 4.9, 1.1 Hz, 1H), 3.72 (ddd, J=8.1, 7.3, 5.9 Hz, 2H), 2.40 (s, 3H), 1.73 (dt, J=14.7, 7.3 Hz, 2H), 1.02 (t, J=7.4 Hz, 3H). ¹³C NMR (101 MHZ, CDCl₃) δ 177.95, 152.87, 148.02, 137.37, 123.92, 123.56, 45.88, 22.55, 22.28, 11.43.

Example 18

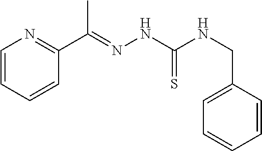 [SMILES: C/C(=N\NC(=S)NCc1ccccc1)c1ccccn1]

(E)-N-benzyl-2-(1-(pyridin-2-yl)ethylidene)hydrazine-1-carbothioamide (Compound 18)

Following General Method A, compound 18 was isolated as a white solid. Yield 46%. ¹H NMR (400 MHZ, CDCl₃) δ 8.80 (s, 1H), 8.62 (ddd, J=4.8, 1.8, 0.9 Hz, 1H), 7.89 (dt, J=8.0, 1.1 Hz, 2H), 7.69 (td, J=7.7, 1.8 Hz, 1H), 7.47-7.37 (m, 4H), 7.35 (ddd, J=8.0, 4.4, 1.9 Hz, 1H), 7.31-7.27 (m, 2H), 5.03 (d, J=5.8 Hz, 2H), 2.45 (s, 3H). ¹³C NMR (400 MHZ, CDCl₃) δ 178.35, 155.12, 154.41, 148.90, 148.01, 137.43, 136.31, 128.84, 127.75, 124.05, 120.33, 48.55, 11.48.

### Example 19

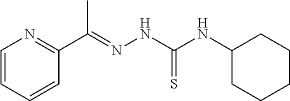 [SMILES: C/C(=N\NC(=S)NC1CCCCC1)c1ccccn1]

(E)-N-cyclohexyl-2-(1-(pyridin-2-yl)ethylidene)hydrazine-1-carbothio amide (Compound 19)

Following General Method A, compound 19 was isolated as a white solid. Yield 52%. ¹H NMR (400 MHZ, CDCl₃) δ 8.66-8.60 (m, 2H), 7.94 (dd, J=8.1, 1.1 Hz, 1H), 7.74 (td, J=7.8, 1.8 Hz, 1H), 7.52 (d, J=8.6 Hz, 1H), 7.31 (ddd, J=7.5, 4.9, 1.2 Hz, 1H), 4.35 (dddd, J=10.4, 8.5, 6.4, 4.3 Hz, 1H), 2.44-2.41 (m, 3H), 2.22-2.13 (m, 2H), 1.79 (dt, J=13.3, 4.0 Hz, 2H), 1.74-1.65 (m, 2H), 1.55-1.43(m, 2H), 1.41-1.30 (m, 2H). ¹³C NMR (101 MHZ, CDCl₃) δ 176.59, 154.65, 148.90, 147.36, 136.26, 123.92, 120.15, 53.18, 32.67, 25.53, 24.78, 11.38.

### Example 20

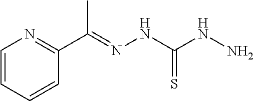

### (E)-N′-(1-(pyridin-2-yl)ethylidene)hydrazinecarbothiohydrazide (Compound 20)

To a solution of 2-acetylpyridine (1.0 mmol) and thiocarbohydrazide (1.0 mmol) in EtOH (1 ml) was added AcOH (1 drop). After stirring at reflux overnight, the solution was cooled to 5° C. and compound 20 was isolated as a white solid (115 mg, 55%). ¹H NMR (400 MHZ, DMSO-d₆) δ 10.33 (s, 1H), 9.98 (s, 1H), 8.60-8.51 (m, 2H), 7.79 (td, J=7.8, 1.9 Hz, 1H), 7.37 (ddd, J=7.4, 4.8, 1.1 Hz, 1H), 4.99 (s, 2H), 2.37 (s, 3H). ¹³C NMR (101 MHZ, DMSO-d₆) δ 181.98, 176.66, 155.19, 136.71, 124.28, 121.54, 39.58, 12.45.

### Example 21

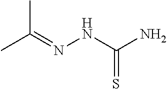

2-(propan-2-ylidene)hydrazine-1-carbothioamide (Compound 21)

Following Example 11, compound 21 was isolated as a white solid (26 mg, 20%). ¹H NMR (400 MHz, DMSO-d₆) δ 9.92 (s, 1H), 8.00 (s, 1H), 7.52 (s, 1H), 1.92 (d, J=7.3 Hz, 6H). ¹³C NMR (101 MHz, DMSO-d₆) δ 178.85, 152.06, 25.50, 18.02.

### Example 22

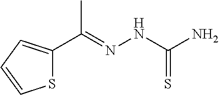

(E)-2-(1-(thiophen-2-yl)ethylidene)hydrazine-1-carbothioamide (Compound 22)

Following Example 11, compound 22 was isolated as a yellow solid (76 mg, 38%). ¹H NMR (400 MHz, CDCl₃) δ 8.79 (s, 1H), 7.36 (dd, J=15.7, 4.3 Hz, 3H), 7.06 (dd, J=5.0, 3.7 Hz, 1H), 6.55 (s, 1H), 2.33 (s, 3H). ¹³C NMR (101 MHz, CDCl₃) δ 178.82, 143.85, 142.23, 128.59, 127.82, 127.60, 13.86.

### Example 23

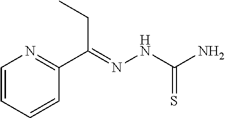

(E)-2-(1-(pyridin-2-yl)propylidene)hydrazine-1-carbothioamide (Compound 23)

Following Example 11, compound 23 was isolated as a light brown solid (121 mg, 58%). ¹H NMR (400 MHZ, DMSO-d₆) δ 10.51 (s, 1H), 8.60-8.55 (m, 1H), 8.42 (d, J=8.1 Hz, 2H), 8.14 (s, 1H), 7.78 (td, J=7.8, 1.8 Hz, 1H), 7.42-7.35 (m, 1H), 3.05 (q, J=7.4 Hz, 2H), 1.00 (t, J=7.4 Hz, 3H). ¹³C NMR (101 MHz, DMSO-d₆) δ 179.54, 178.81, 154.54, 152.44, 152.32, 148.93, 148.51, 143.05, 138.94, 136.88, 125.13, 124.60, 124.33, 121.72, 28.04, 18.22, 12.29, 11.24.

### Example 24

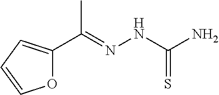

(E)-2-(1-(furan-2-yl)ethylidene)hydrazine-1-carbothioamide (Compound 24) Following Example 11, compound 24 was isolated as a yellow solid (78 mg, 43%). ¹H NMR (400 MHz, DMSO-d₆) δ 10.31 (s, 1H), 8.31 (s, 1H), 7.77 (dd, J=1.8, 0.8 Hz, 1H), 7.72 (s, 1H), 7.12 (dd, J=3.5, 0.8 Hz, 1H), 6.60 (dd, J=3.5, 1.8 Hz, 1H), 2.24 (s, 3H). ¹³C NMR (101 MHZ, DMSO-d₆) δ 179.09, 152.23, 144.76, 140.56, 112.63, 111.09, 13.79.

### Example 25

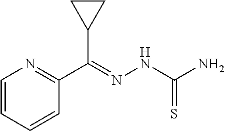 [SMILES: NC(=S)N/N=C(/c1ccccn1)C1CC1]

(E)-2-(cyclopropyl(pyridin-2-yl)methylene)hydrazine-1-carbothioamide (Compound 25)

Following Example 11, compound 25 was isolated as a brown solid (141 mg, 64%). 1H NMR (400 MHz, CDCl₃) δ 14.51 (s, 1H), 8.77 (ddd, J=4.9, 1.8, 0.9 Hz, 1H), 8.00 (dt, J=8.1, 1.1 Hz, 1H), 7.92 (td, J=7.9, 1.9 Hz, 1H), 7.41 (ddd, J=7.6, 4.9, 1.2 Hz, 1H), 7.35 (s, 1H), 6.26 (s, 1H), 1.99 (tt, J=7.8, 5.4 Hz, 1H), 0.97 (tq, J=6.4, 1.9 Hz, 4H). ¹³C NMR (101 MHz, CDCl₃) δ 179.34, 153.20, 147.98, 142.71, 137.52, 124.19, 123.73, 15.25, 7.14.

### Example 26

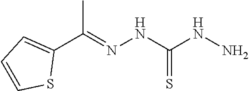

### (E)-N′-(1-(thiophen-2-yl)ethylidene)hydrazinecarbothiohydrazide (Compound 26)

Following Example 20, compound 26 was isolated as a yellow solid (139 mg, 65%). ¹H NMR (400 MHz, DMSO-d₆) δ 10.36 (s, 1H), 9.21 (s, 1H), 7.60 (dd, J=5.0, 1.2 Hz, 1H), 7.56-7.52 (m, 1H), 7.09 (dd, J=5.1, 3.7 Hz, 1H), 4.92 (s, 2H), 2.32 (s, 3H). ¹³C NMR (101 MHz, DMSO-d₆) δ 176.83, 145.54, 143.29, 129.03, 128.35, 128.13, 15.24.

### Example 27

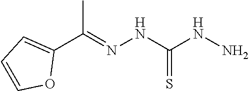

### (E)-N′-(1-(furan-2-yl)ethylidene)hydrazinecarbothiohydrazide (Compound 27)

Following Example 20, compound 27 was isolated as a light yellow solid (115 mg, 58%). ¹H NMR (400 MHZ, DMSO-d₆) δ 10.31 (s, 1H), 9.49 (s, 1H), 7.76 (dd, J=1.7, 0.8 Hz, 1H), 7.18 (d, J=3.5 Hz, 1H), 6.60 (dd, J=3.4, 1.8 Hz, 1H), 4.92 (s, 2H), 2.22 (s, 3H). ¹³C NMR (101 MHz, DMSO-d₆) δ 176.71, 152.35, 144.57, 140.65, 112.65, 110.72, 13.60.

### Example 28

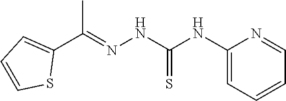 [SMILES: C/C(=N\NC(=S)Nc1ccccn1)c1cccs1]

(E)-N-(pyridin-2-yl)-2-(1-(thiophen-2-yl)ethylidene)hydrazine-1-carbothioamide (Compound 28)

General Method D: To a cooled solution of NaOH (3.0 mol) in 2.4 mL of water and 2.0 mL of 2-propanol was added hydrazine hydrate (0.03 mol). Ice-cooled carbon disulfide (0.03 mol) was added dropwise to the stirred solution, which was maintained at <10° C. The bright-yellow mixture was stirred for an additional 1 h, after which ice-cooled iodomethane (0.03 mol) was added dropwise over 0.5 h. After stirring for an additional 1 h, the white precipitate was collected and recrystallized from CH2Cl2 to give the intermediate methyl hydrazinecarbodithioate. Methyl hydrazinecarbodithioate (0.02 mol) and 2-acetylpyridine (0.02 mol) in 5 mL of EtOH were stirred under reflux overnight. The precipitate was collected to give the intermediate methyl 3-[1-(2-pyridyl)ethylidene]hydrazinecarbodithioate. To 1 mmol of methyl 3-[1-(2-pyridyl)ethylidene]hydrazinecarbodithioate dissolved in 0.5 mL of EtOH was added 0.01 mmol of 2-aminopyridine. After stirring at reflux overnight, the solution was concentrated and purified via chromatography on silica gel to afford compound 28 as a white solid (118 mg, 43%). ¹H NMR (400 MHz, DMSO-d₆) δ 14.88 (s, 1H), 10.97 (s, 1H), 8.35 (d, J=5.1 Hz, 1H), 7.96-7.79 (m, 1H), 7.61 (dd, J=23.6, 4.3 Hz, 3H), 7.28 (d, J=8.3 Hz, 1H), 7.21-7.01 (m, 2H), 2.45 (s, 4H). ¹³C NMR (101 MHz, DMSO-d₆) δ 177.40, 153.83, 150.08, 146.18, 143.23, 139.94, 129.87, 128.86, 128.05, 118.43, 113.25, 15.72.

### Example 29

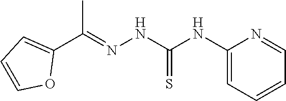 [SMILES: C/C(=N\NC(=S)Nc1ccccn1)c1ccco1]

(E)-2-(1-(furan-2-yl)ethylidene)-N-(pyridin-2-yl)hydrazine-1-carbothioamide (Compound 29)

Following General Method D, compound 29 was isolated as a yellow solid (106 mg, 41%). ¹H NMR (400 MHZ, DMSO-d₆) δ 14.86 (s, 1H), 10.98 (s, 1H), 8.35 (s, 2H), 7.86 (q, J=6.5, 4.0 Hz, 3H), 7.39-7.00 (m, 5H), 6.72-6.56 (m, 1H), 2.35 (s, 4H). ¹³C NMR (101 MHz, DMSO-d₆) δ 177.69, 153.84, 151.82, 146.31, 145.54, 145.45, 139.96, 118.43, 113.28, 112.75, 112.60, 14.96.

### Example 30

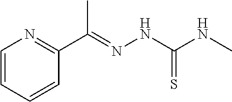

(E)-N-methyl-2-(1-(pyridin-2-yl)ethylidene)hydrazine-1-carbothioamide (Compound 30)

Following General Method C, compound 30 was isolated as a white solid (148 mg, 71%). ¹H NMR (400 MHZ, CDCl₃) δ 8.78 (s, 1H), 8.62 (ddd, J=4.8, 1.8, 1.0 Hz, 1H), 7.98 (dt, J=8.1, 1.1 Hz, 1H), 7.73 (td, J=7.8, 1.8 Hz, 1H), 7.67 (s, 1H), 7.34-7.29 (m, 1H), 3.30 (d, J=4.9 Hz, 3H), 2.42 (s, 3H). ¹³C NMR (101 MHZ, CDCl₃) δ 178.98, 154.57, 148.91, 147.61, 136.26, 123.98, 120.29, 31.36, 11.43.

Example 31

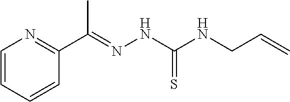 [SMILES: C=CCNC(=S)N/N=C(\C)c1ccccn1]

(E)-N-allyl-2-(1-(pyridin-2-yl)ethylidene)hydrazine-1-carbothioamide (Compound 31)

Following General Method C, compound 31 was isolated as a yellow solid (159 mg, 68%). ¹H NMR (400 MHZ, CDCl₃) δ 8.86-8.69 (m, 1H), 8.63 (ddd, J=4.9, 1.8, 0.9 Hz, 1H), 7.97 (dt, J=8.1, 1.1 Hz, 1H), 7.80-7.60 (m, 2H), 7.36-7.29 (m, 1H), 6.01 (ddt, J=17.2, 10.2, 5.6 Hz, 1H), 5.41-5.20 (m, 1H), 4.43 (tt, J=5.8, 1.6 Hz, 2H), 2.44 (s, 2H). ¹³C NMR (101 MHz, CDCl₃) δ 178.21, 154.50, 148.91, 147.88, 136.31, 133.29, 124.03, 120.28, 117.14, 46.99, 11.46.

### Example 32

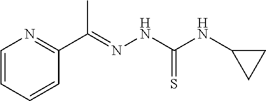 [SMILES: C/C(=N\NC(=S)NC1CC1)c1ccccn1]

(E)-N-cyclopropyl-2-(1-(pyridin-2-yl)ethylidene)hydrazine-1-carbothio amide (Compound 32)

Following General Method D, compound 31 was isolated as a white solid (131 mg, 56%). ¹H NMR (400 MHZ, CDCl₃) δ 8.76 (s, 1H), 8.62 (ddd, J=4.8, 1.8, 0.9 Hz, 1H), 7.92 (dt, J=8.1, 1.1 Hz, 1H), 7.74 (ddd, J=8.2, 7.5, 1.8 Hz, 1H), 7.63 (s, 1H), 7.31 (ddd, J=7.5, 4.9, 1.2 Hz, 1H), 3.18 (tq, J=7.2,3.7 Hz, 1H), 2.42 (s, 3H), 1.03-0.91 (m, 2H), 0.82-0.71 (m, 2H). ¹³C NMR (101 MHZ, CDCl₃) δ 179.66, 154.49, 148.92, 147.66, 136.29, 124.01, 120.27, 77.35, 27.04, 11.45, 7.54.

### Example 33

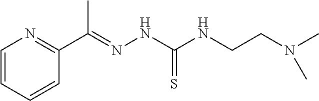 [SMILES: C/C(=N\NC(=S)NCCN(C)C)c1ccccn1]

(E)-N-(2-(dimethylamino)ethyl)-2-(1-(pyridin-2-yl)ethylidene)hydrazine-1-carbothioamide (Compound 33)

Following General Method D, compound 33 was isolated as a brown solid (108 mg, 41%). ¹H NMR (400 MHZ, CDCl₃) δ 8.75 (s, 1H), 8.62 (ddd, J=4.9, 1.9, 1.0 Hz, 1H), 8.40 (s, 1H), 8.08 (dt, J=8.1, 1.1 Hz, 1H), 7.73 (td, J=7.8, 1.8 Hz, 1H), 7.35-7.30 (m, OH), 3.92-3.82 (m, 2H), 2.73 (t, J=6.0 Hz, 2H), 2.41 (d, J=10.0 Hz, 10H). ¹³C NMR (101 MHZ, CDCl₃) δ 177.82, 154.65, 148.74, 147.29, 136.27, 123.87, 120.38, 57.22, 44.95, 41.75, 11.15.

### Example 34

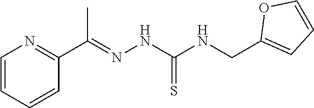 [SMILES: C/C(=N\NC(=S)NCc1ccco1)c1ccccn1]

(E)-N-(furan-2-ylmethyl)-2-(1-(pyridin-2-yl)ethylidene)hydrazine-1-carbothioamide (Compound 34)

Following General Method D, compound 34 was isolated as a light yellow solid (140 mg, 51%). ¹H NMR (400 MHZ, CDCl₃) δ 8.82 (s, 1H), 8.61 (dd, J=4.8, 2.1 Hz, 1H), 8.00-7.86 (m, 2H), 7.72 (tt, J=7.8, 2.1 Hz, 1H), 7.31 (ddt, J=9.6, 6.1, 1.7 Hz, 1H), 6.39 (dd, J=4.7, 2.4 Hz, 2H), 4.99 (dd, J=5.6, 2.1 Hz, 2H), 2.42 (s, 2H). ¹³C NMR (101 MHZ, CDCl₃) δ 178.04, 154.39, 150.36, 148.89, 148.09, 142.49, 136.35, 124.09, 120.39, 110.57, 108.29, 41.50, 11.51.

### Example 35

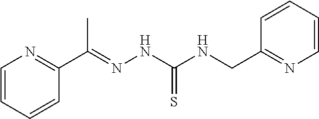 [SMILES: C/C(=N\NC(=S)NCc1ccccn1)c1ccccn1]

(E)-2-(1-(pyridin-2-yl)ethylidene)-N-(pyridin-2-ylmethyl)hydrazine-1-carbothioamide (Compound 35)

Following General Method D, compound 35 was isolated as a yellow solid (88 mg, 31%). ¹H NMR (400 MHZ, CDCl₃) δ 9.03 (s, 1H), 8.85 (s, 1H), 8.62 (ddt, J=4.4, 2.5, 1.1 Hz, 2H), 8.15 (dt, J=8.2, 1.1 Hz, 1H), 7.83-7.67 (m, 2H), 7.41 (dt, J=7.8, 1.0 Hz, 1H), 7.36-7.24 (m, 3H), 5.07 (d, J=4.9 Hz, 2H), 2.45 (s, 3H). ¹³C NMR (101 MHz, CDCl₃) δ 177.85, 155.69, 154.62, 149.11, 148.77, 147.55, 136.86, 136.31, 123.93, 122.58, 122.26, 120.45, 49.14, 11.21.

### Example 36

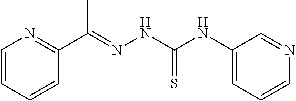 [SMILES: C/C(=N\NC(=S)Nc1cccnc1)c1ccccn1]

(E)-2-(1-(pyridin-2-yl)ethylidene)-N-(pyridin-3-yl)hydrazine-1-carbothioamide (Compound 36)

Following General Method D, compound 36 was isolated as a yellow solid (124 mg, 46%). ¹H NMR (400 MHZ, CDCl₃) δ 9.45 (s, 1H), 9.00 (s, 1H), 8.72 (d, J=2.6 Hz, 1H), 8.66 (ddd, J=4.8, 1.8, 0.9 Hz, 1H), 8.50 (dd, J=4.7, 1.5 Hz, 1H), 8.40 (ddd, J=8.3, 2.7, 1.5 Hz, 1H), 8.03 (dt, J=8.1, 1.1 Hz, 1H), 7.78 (td, J=7.7, 1.8 Hz, 1H), 7.41-7.33 (m, 2H), 2.52 (s, 3H). ¹³C NMR (101 MHz, CDCl₃) δ 176.78, 154.10, 149.09, 148.87, 147.07, 145.32, 136.51, 134.75, 131.53, 124.40, 123.21, 120.45, 11.85.

### Example 37

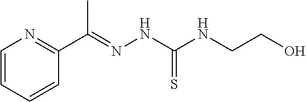 [SMILES: C/C(=N\NC(=S)NCCO)c1ccccn1]

(E)-N-(2-hydroxyethyl)-2-(1-(pyridin-2-yl)ethylidene)hydrazine-1-carbothioamide (Compound 37)

Following General Method D, compound 37 was isolated as a white solid (111 mg, 47%). ¹H NMR (400 MHZ, CDCl₃) δ 8.81 (s, 1H), 8.62 (dt, J=4.8, 1.5 Hz, 1H), 8.22 (s, 1H), 7.94 (dt, J=8.1, 1.1 Hz, 1H), 7.74 (td, J=7.8, 1.8 Hz, 1H), 7.31 (ddd, J=7.5, 4.8, 1.2 Hz, 1H), 3.95 (d, J=2.4 Hz, 4H), 3.04 (d, J=4.8 Hz, 1H), 2.40 (s, 3H). ¹³C NMR (101 MHz, CDCl₃) δ 178.49, 154.32, 148.91, 147.46, 136.56, 124.09, 120.64, 61.58, 46.80, 11.58.

### Example 38

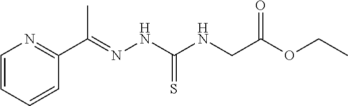 [SMILES: CCOC(=O)CNC(=S)N/N=C(\C)c1ccccn1]

ethyl (E)-(2-(1-(pyridin-2-yl)ethylidene)hydrazine-1-carbonothioyl)glycinate (Compound 38)

Following General Method D, compound 38 was isolated as a brown solid (120 mg, 43%). ¹H NMR (400 MHZ, CDCl₃) δ 8.87 (s, 1H), 8.62 (ddd, J=4.9, 1.8, 1.0 Hz, 1H), 8.17 (s, 1H), 8.06 (dt, J=8.1, 1.1 Hz, 1H), 7.75 (ddd, J=8.1, 7.5, 1.8 Hz, 1H), 7.32 (ddd, J=7.5, 4.9, 1.2 Hz, 1H), 4.54 (d, J=4.9 Hz, 2H), 4.31 (q, J=7.1 Hz, 2H), 2.44 (s, 2H), 1.36 (t, J=7.1 Hz, 2H). ¹³C NMR (101 MHZ, CDCl₃) δ 178.14, 169.46, 154.36, 148.82, 148.18, 136.42, 124.09, 120.39, 61.82, 46.20, 14.18, 11.34.

### Example 39

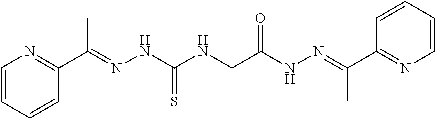 [SMILES: C/C(=N\NC(=O)CNC(=S)N/N=C(\C)c1ccccn1)c1ccccn1]

(E)-N-(2-oxo-2-(2-((E)-1-(pyridin-2-yl)ethylidene)hydrazinyl)ethyl)-2-(1-(pyridin-2-yl)ethylidene)hydrazine-1-carbothioamide (Compound 39)

Following General Method A, compound 39 was isolated as a white solid (313 mg, 85%). ¹H NMR (400 MHZ, DMSO-d₆) δ 10.99 (s, 1H), 10.66 (s, 1H), 8.87 (t, J=5.8 Hz, 1H), 8.62 (qd, J=3.0, 2.5, 1.0 Hz, 3H), 8.36 (t, J=7.5 Hz, 1H), 8.08 (d, J=8.2 Hz, 1H), 7.87 (qd, J=7.8, 1.8 Hz, 3H), 7.51-7.32 (m, 3H), 4.87 (d, J=5.7 Hz, 2H), 2.44 (s, 4H), 2.37 (s, 3H). ¹³C NMR (101 MHz, DMSO-d₆) δ 179.34, 171.25, 155.37, 155.15, 149.24, 149.08, 148.79, 137.19, 136.88, 124.49, 124.42, 120.99, 120.76, 120.31, 46.11, 12.84, 12.31.

### Example 40

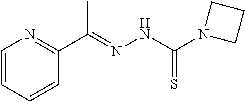 [SMILES: C/C(=N\NC(=S)N1CCC1)c1ccccn1]

(E)-N′-(1-(pyridin-2-yl)ethylidene)azetidine-1-carbothiohydrazide (Compound NSC)

Following General Method A, compound NSC was isolated as a white solid. Yield 41%. ¹H NMR (400 MHZ, CDCl₃) δ 8.74 (s, 1H), 8.61 (ddd, J=5.0, 1.9, 1.0 Hz, 1H), 7.87 (dt, J=8.2, 1.1 Hz, 1H), 7.70 (td, J=7.8, 1.7 Hz, 1H), 7.37-7.20 (m, 1H), 4.72 (d, J=8.1 Hz, 2H), 4.37 (t, J=7.9 Hz, 2H), 2.57-2.29 (m, 5H).

### Biological Examples

### High Throughput Screening of NMNAT2 Activators by Using Doubly Coupled NMNAT2 Assay

A high-throughput screen was performed in search of compounds capable of activating NMNAT2, an important NAD biosynthetic enzyme. Roughly 50,000 synthetic organic chemicals were tested in a doubly coupled NMNAT assay that contains two enzymes, NMNAT2 and alcohol dehydrogenase (ADH). NMNAT2 facilitates conversion of NMN into NAD, and ADH facilitates conversion of NAD into the fluorescent end product, NADH. The NMNAT2 enzyme reaction was prepared to contain 50 mM Tris-Cl (pH 8.0), 12 mM MgCl₂, 1.5% ethanol, 2.5 mM ATP, 10 mM semicarbizide, 0.2% bovine serum albumin (BSA), 0.1 μg/ml NMNAT2, and 0.4 units ADH. 40 □1 of the mixture was dispensed into 384-well black polypropylene plates, and individual compounds were added to each well at a final concentration of 5 μM. The reaction was initiated by addition of nicotinamide mononucleotide (NMN) at 50 μM followed by gentle mixing in the plate. The conversion of NMN to NADH was monitored by measuring fluorescence intensity of NADH at an excitation of 340 nm and an emission of 445 nm (Y axis) as a function of time (X axis). Fluorescence at ex340 nm/em445 nm (F_{340/445}) was measured continuously for 30 minutes at room temperature to establish the reaction velocity. FIG. 2 is a representative plot for the kinetic NMNAT2 reaction in the presence and absence of compound NSC. The reaction rate is expressed as the concentration of NADH (equivalent to NAD) formed per minute. Since the concentration of NADH is reflected by F_{340/445}, the reaction rate is thus the slope of the linear kinetic curve of F_{340/445} versus time at time t, written as F340 445/min, which is calculated in the equation: reaction rate=ΔF_{340/445}/Δt. The relative NMNAT2 enzyme reaction rate in the presence of each compound is normalized by DMSO-treated control.

In a secondary screen, the compounds with the relative reaction rate higher than 150% are cherry-picked and retested in triplicates in the doubly coupled NMNAT2 assay. The compounds exhibiting activating properties were re-tested in an ADH enzyme assay that used NAD as a substrate. Briefly, the ADH enzyme reaction was prepared to contain 50 mM Tris-Cl (pH 8.0), 12 mM MgCl₂, 1.5% ethanol, 10 mM semicarbizide, 0.2% bovine serum albumin (BSA), and 0.04 units ADH. 40 □l of the mixture was dispensed into 384-well black polypropylene plates, and individual compounds were added to each well at a final concentration of 5 μM. The reaction was initiated by addition of 50 μM NAD followed by gentle mixing in the plate. The conversion of NAD to NADH was monitored by measuring fluorescence intensity of NADH at an excitation of 340 nm and an emission of 445 nm (Y axis) as a function of time (X axis). Fluorescence at ex340 nm/em445 nm was measured continuously for 30 minutes to establish the kinetic reaction curve of F_{340/445} versus time. The reaction rates were calculated as described in the NMNAT2 assay. The relative ADH enzyme reaction rate in the presence of each compound is normalized by DMSO-treated control.

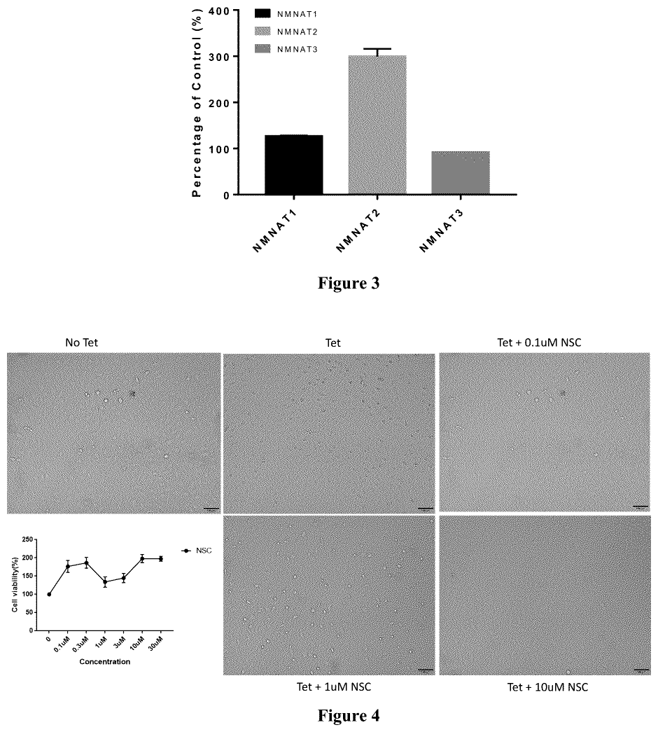

Some compounds were observed to be active in the doubly coupled enzyme assay, but inactive in the ADH assay. These compounds might represent direct activators of the NMNAT2 enzyme. Among them, compound NSC, a pyridine semicarbazone ((E)-N′-(1-(pyridin-2-yl)ethylidene)azetidine-1-carbothio hydrazide) exhibited the most robust level of activation in the doubly coupled NMNAT2 assay (FIG. 2). Intriguingly, subsequent test of the effect of compound NSC on the other two NMNAT isoforms revealed that the compound activated NMNAT2 strongly and NMNAT1 weakly, but not NMNAT3 (FIG. 3).

### Activity Assays of Compounds NSC and 1-40

Compounds NSC and 1-39 were synthesized according to the Preparation Examples of the present disclosure. Compound 40 was purchased from Selleck.

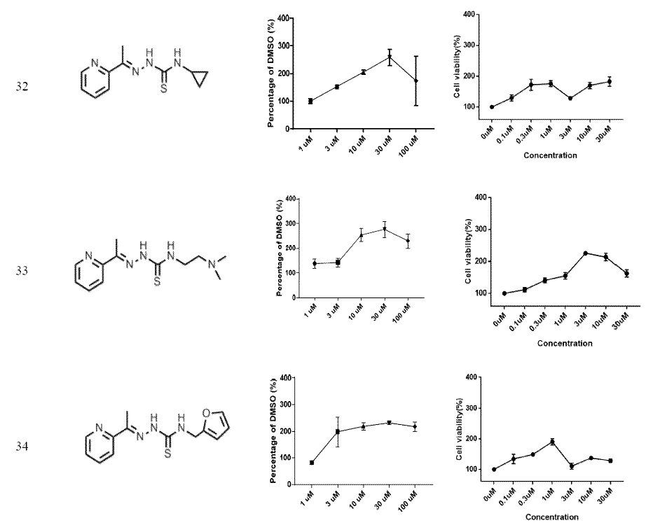

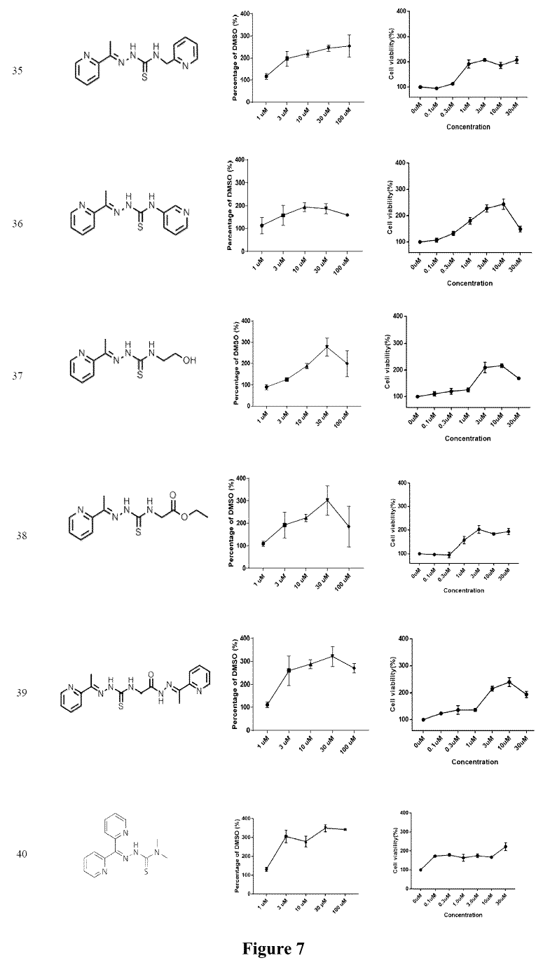

Compounds NSC and 1-40 were evaluated in two independent assays: an in vitro enzyme assay measuring activation of NMNAT2 and a cell-based assay measuring the degree of protection from SARM1 expression in cells. In the first assay, the compounds were added at 1, 3, 10, 30 and 100 μM in the reaction mixture (50 mM Tris-Cl (pH 8.0), 12 mM MgCl₂, 1.5% ethanol, 2.5 mM ATP, 10 mM semicarbizide, 0.2% bovine serum albumin (BSA), 0.1 μg/ml NMNAT2, and 0.4 units ADH). The reaction was initiated by addition of nicotinamide mononucleotide (NMN) at 50 μM followed by gentle mixing. The conversion of NMN to NADH was monitored by measuring fluorescence intensity of NADH at an excitation of 340 nm and an emission of 445 nm (Y axis) as a function of time (X axis). Fluorescence at ex340 nm/em445 nm (F_{340/445}) was measured continuously for 30 minutes at room temperature to establish the reaction velocity. The reaction rate of NMNAT2 enzyme was expressed as the concentration of NADH (equivalent to NAD) formed per minute. Since the concentration of NADH is reflected by F_{340/445}, the reaction rate is thus the slope of the linear kinetic curve of F_{340/445} versus time at time t, written as F_{340/445}/min, which is calculated in the equation: reaction rate=ΔF_{340/445}/Δt. The relative NMNAT2 enzyme reaction rate in the presence of each compound is normalized by DMSO-treated control. The dose response curves were plotted to assess the effects of individual compounds on NMNAT2 enzymatic activity. In the second assay, SARM1-expressing cells were treated with the compounds at 0.1, 0.3, 1, 3, 10, and 30 μM. After 24 hours, the cell survival was determined by measuring ATP levels using Cell-titer Glo assay kit, and normalized by DMSO-treated control. The dose response curves were plotted to evaluate the cell-protecting activity of the compounds. The results of the two assays are shown in FIG. 7.

### In Vivo Neuroprotective Effects in Mouse CIPN Model

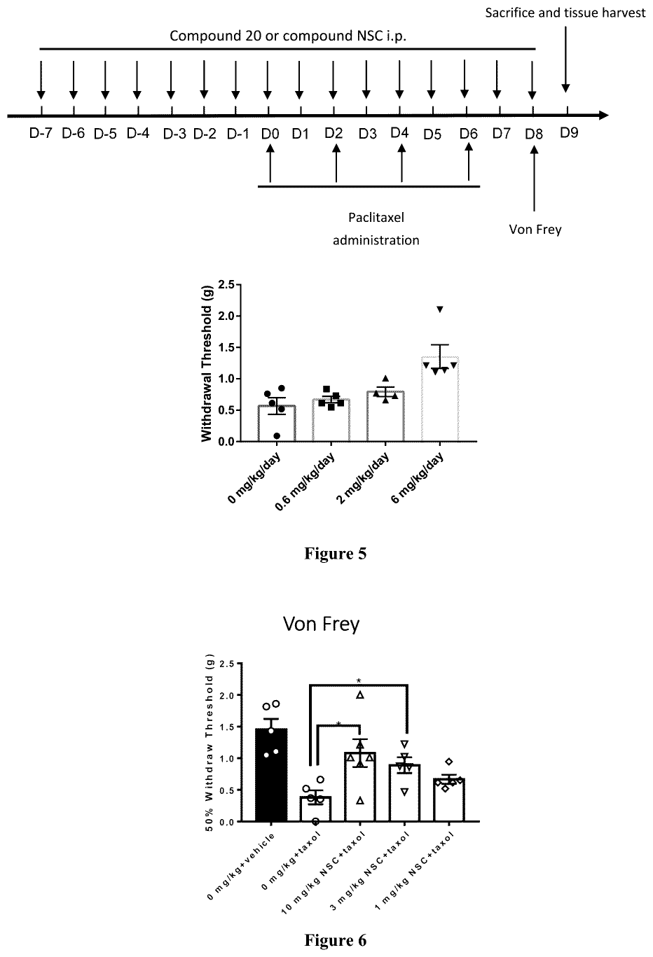

Chemotherapy-induced peripheral neuropathy (CIPN) arises from the peripheral nerve damage by anticancer pharmacotherapy, leading to a progressive, enduring, and debilitating condition featuring pain, numbness, tingling and sensitivity to cold in the hands and feet. CIPN afflicts between 30% and 40% of patients undergoing chemotherapy, but currently no effective treatment for CIPN exists (del Pino BM, 2010). Chemotherapy drugs associated with CIPN include the antimitotic drugs such as paclitaxel and vinblastine, which are widely used for the treatment of a variety of cancer. To evaluate the neuroprotective efficacy of the compounds in vivo, we developed an aggressive CIPN mice model as presented in FIG. 5.

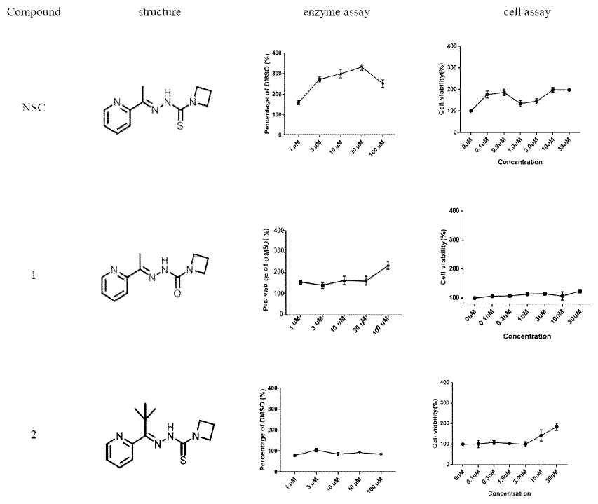

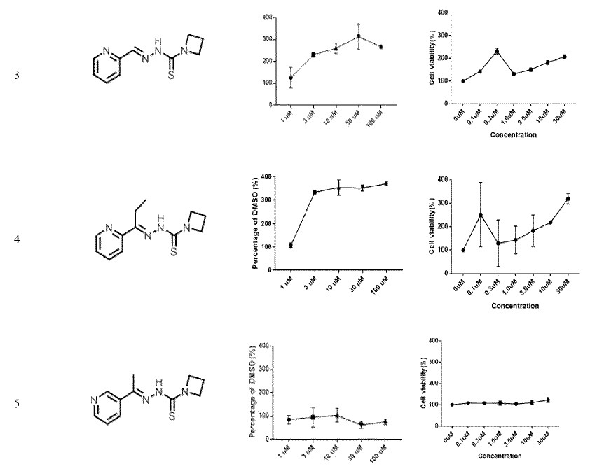

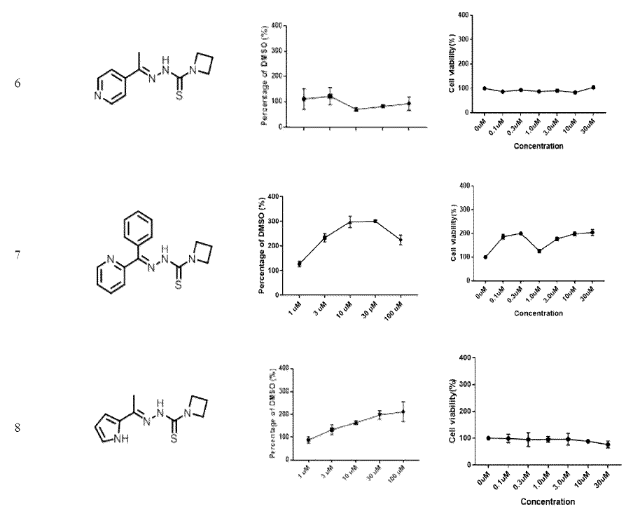

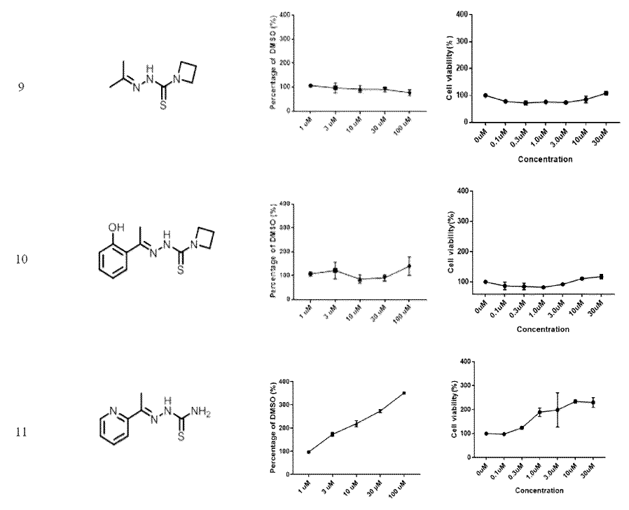

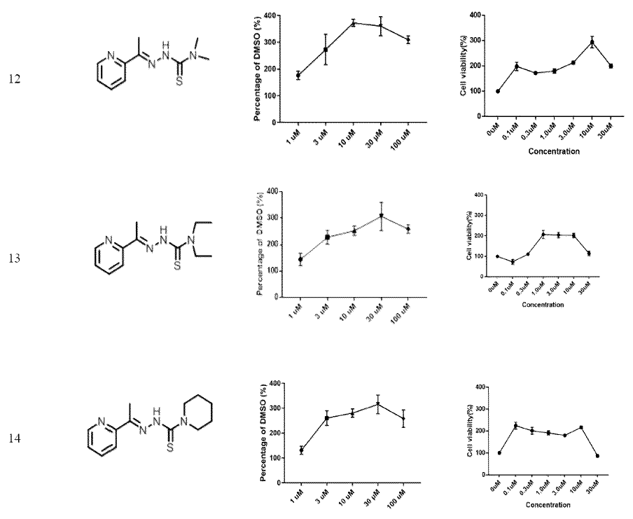

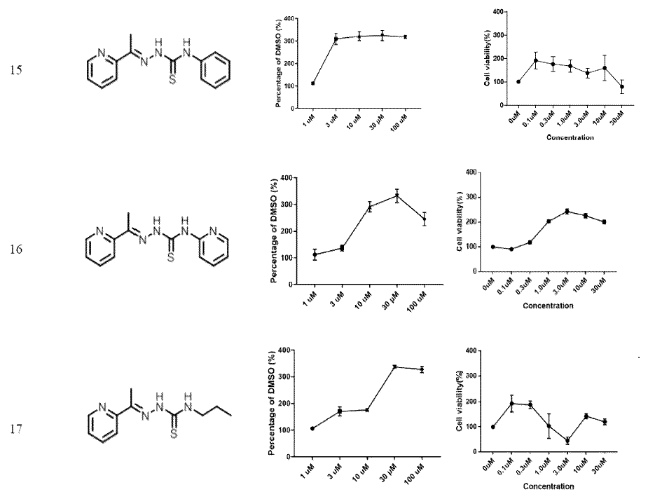

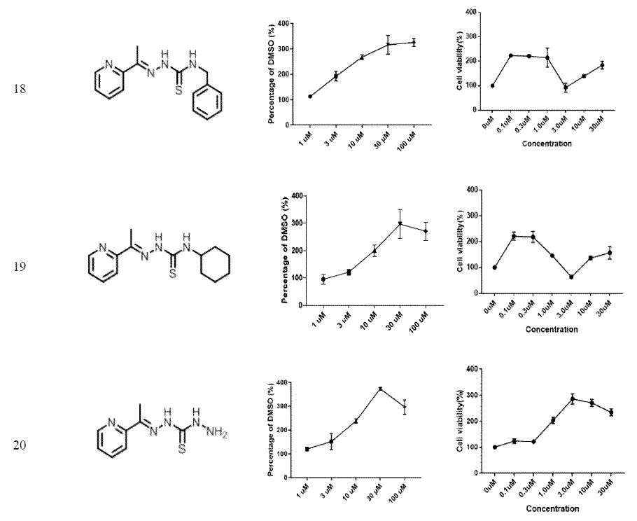

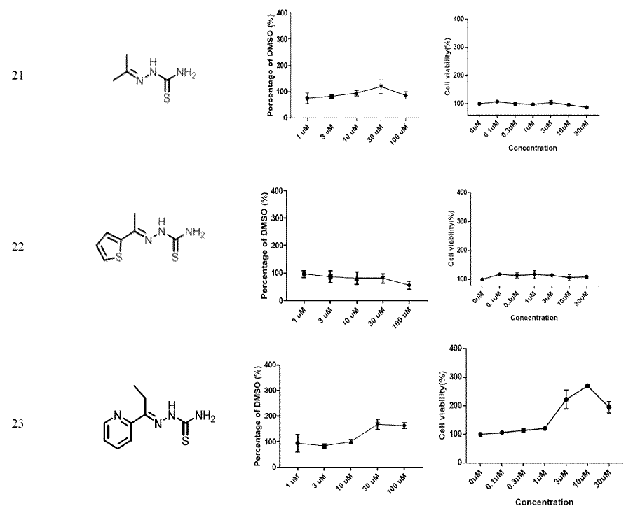

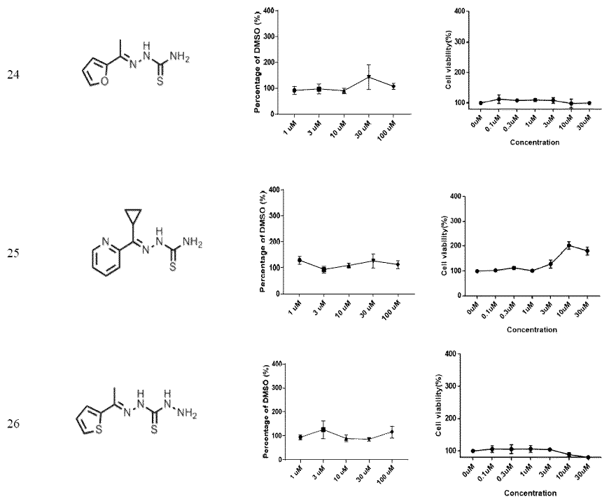

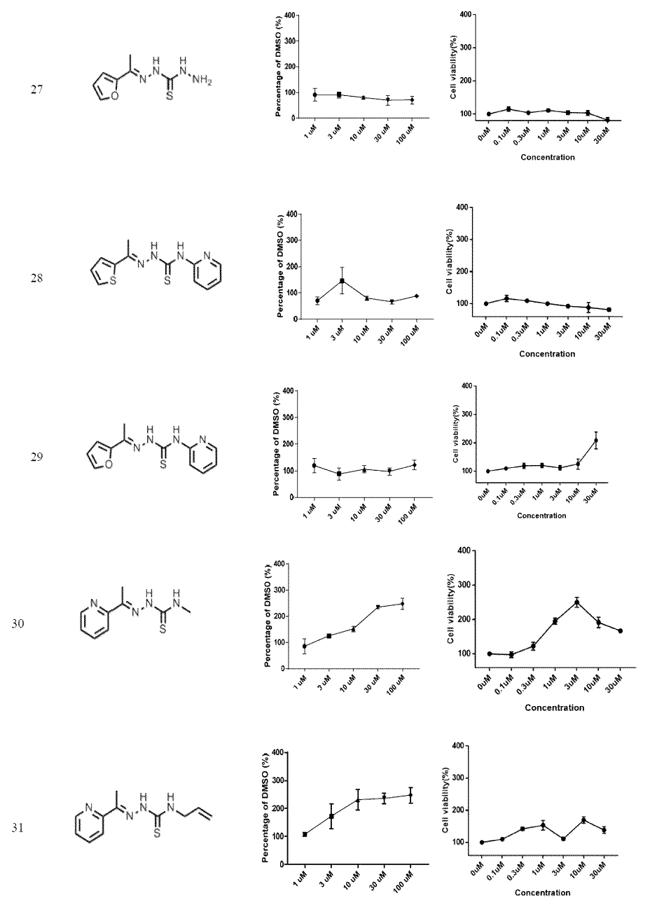

Mice were daily administrated with compound 20, at doses of 6, 2, 0.6 and 0 mg/kg (5 animals per group, i.p.) from one week prior to the initiation of paclitaxel (D-7) to 8^{th} day after the initiation of paclitaxel (D8). After one-week pre-dosing with compound 20, mice were injected intraperitoneally with paclitaxel at a dose of 2 mg/kg on 4 alternate days (Day 0, 2, 4, 6). The Von Frey test was conducted the other day after the last administration of paclitaxel. In the CIPN mice model, compound 20 significantly increased the mice paw pressure threshold at a dose of 6 mg/kg/day i.p. (FIG. 5). Mice were daily administrated with compound NSC, at doses of 1, 3, and 10 mg/kg (5-6 animals per group, i.p.) from one week prior to the initiation of taxol (paclitaxel) (D-7) to 8^{th} day after the initiation of paclitaxel (D8). After one-week pre-dosing with compound NSC, mice were injected intraperitoneally with paclitaxel at a dose of 2 mg/kg on 4 alternate days (Day 0, 2, 4, 6). The Von Frey test was conducted the other day after the last administration of taxol (paclitaxel). In the CIPN mice model, compound NSC significantly increased the mice paw pressure threshold at a dose of 10 or 3 mg/kg/day i.p. (FIG. 6).

### REFERENCES

1. Cambronne, X. A., Stewart, M. L., Kim, D., Jones-Brunette, A. M., Morgan, R. K., Farrens, D. L., Cohen, M. S., and Goodman, R. H. (2016). Biosensor reveals multiple sources for mitochondrial NAD (+). Science 352, 1474-1477.
2. Ryu, K. W., Nandu, T., Kim, J., Challa, S., DeBerardinis, R. J., and Kraus, W. L. (2018). Metabolic regulation of transcription through compartmentalized NAD(+) biosynthesis. Science 360.
3. Perrault, I., Hanein, S., Zanlonghi, X., Serre, V., Nicouleau, M., Defoort-Delhemmes, S., Delphin, N., Fares-Taie, L., Gerber, S., Xerri, O., et al. (2012). Mutations in NMNAT1 cause Leber congenital amaurosis with early-onset severe macular and optic atrophy. Nat Genet 44, 975-977.
4. del Pino BM. (2010). Chemotherapy-induced Peripheral Neuropathy. NCI Cancer Bulletin. 7 (4), 6.

What is claimed is:

1. Compounds of the general formula 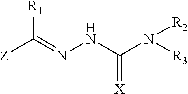
in which
Z represents a heteroaryl group, an aryl group or a C₁-C₆ alkyl group;
R₁ represents hydrogen, a C₁-C₆ alkyl group, a C₃-C₆ cycloalkyl group, or an aryl group;
X represents Se, NH, O or S; and
R₂ or R₃ represents hydrogen, a —OR₄ group, a C₁-C₆ alkyl group, a C₃-C₆ alkenyl group, a C₅-C₈ cycloalkenyl group, a (C₁-C₆ alkyl)-(C₅-C₈ cycloalkenyl) group, a C₃-C₆ alkynyl group, a pyrrolidinyl group, a (C₁-C₆ alkyl)-pyrrolidinyl group, a piperidinyl group, a (C₁-C₆ alkyl)-piperidinyl group, a morpholinyl group, a (C₁-C₆ alkyl)-morpholinyl group, a piperazinyl group, a (C₁-C₆ alkyl)-piperazinyl group, a C₃-C₆ cycloalkyl group, —NR₅R₆, a heteroaryl group, an aryl group, a (C₁-C₆ alkyl)-heteroaryl group, a (C₁-C₆ alkyl)-aryl group, a (C₁-C₆ alkyl)-CO—R₇ group, a (C₁-C₆ alkyl)-NR₅R₆ group, or a (C₁-C₆ alkyl)-OR₄ group; or
R₂ and R₃ together with the nitrogen atom to which they are attached form a 4- to 6-membered heterocycloalkyl;
in which R₄ represents hydrogen, a C₁-C₆ alkyl group, a (C₁-C₆ alkyl)-heteroaryl group or a (C₁-C₆ alkyl)-aryl group;
in which R₅ or R₆ represents hydrogen, a C₁-C₆ alkyl group, a carbonyl group, a sulfoxide group or a sulfone group;
in which R₇ is a —O—(C₁-C₆ alkyl) group, an amino group, a —N—-(C₁-C₆ alkyl) group or a —NH—N═CR₈-heteroaryl group;
in which R₈ is a C₁-C₆ alkyl group;
and the diastereomers, enantiomers, metabolites, salts, solvates thereof or solvates of the salts thereof.

2. The compounds according to claim 1, wherein Z represents pyridyl group, thienyl group, pyrrolyl group, furyl group, hydroxyphenyl group, or a C₁-C₆ alkyl group; more particularly, Z represents pyridin-2-yl group, pyridin-3-yl group, pyridin-4-yl group, thiophen-2-yl group, pyrrol-2-yl group, furan-2-yl group, hydroxyphenyl group, or a C₁-C₆ alkyl group.

3. The compounds according to claim 1, wherein R₁ represents hydrogen, a C₁-C₆ alkyl group, a C₃-C₆ cycloalkyl group, or phenyl group.

4. The compounds according to claim 1, wherein X represents O or S, preferably X represents S.

5. The compounds according to claim 1, wherein
R₂ or R₃ represents hydrogen, a —OR₄ group, a C₁-C₆ alkyl group, a C₃-C₆ alkenyl group, a C₅-C₈ cycloalkenyl group, a (C₁-C₆ alkyl)-(C₅-C₈ cycloalkenyl) group, a C₃-C₆ alkynyl group, a pyrrolidinyl group, a (C₁-C₆ alkyl)-pyrrolidinyl group, a piperidinyl group, a (C₁-C₆ alkyl)-piperidinyl group, a morpholinyl group, a (C₁-C₆ alkyl)-morpholinyl group, a piperazinyl group, a (C₁-C₆ alkyl)-piperazinyl group, a C₃-C₆ cycloalkyl group, —NR₅R₆, pyridyl group, furyl group, phenyl group, a (C₁-C₆ alkyl)-pyridyl group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OR₄ group; or
R₂ and R₃ together with the nitrogen atom to which they are attached form a 4- to 6-membered heterocycloalkyl;
in which R₄ represents hydrogen, a C₁-C₆ alkyl group, a (C₁-C₆ alkyl)-pyridyl group or a (C₁-C₆ alkyl)-phenyl group;
in which R₅ or R₆ represents hydrogen, a C₁-C₆ alkyl group, a carbonyl group, a sulfoxide group or a sulfone group;
in which R₇ is a —O—(C₁-C₆ alkyl) group, an amino group, a —NH—(C₁-C₆ alkyl) group or a —NH—N═CR₈-pyridyl group;
in which R₈ is a C₁-C₆ alkyl group;
particularly,
R₂ or R₃ represents hydrogen, a C₁-C₆ alkyl group, a C₂-C₆ alkenyl group, a C₃-C₆ cycloalkyl group, —NR₅R₆, pyridyl group, furyl group, phenyl group, a (C₁-C₆ alkyl)-pyridyl group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OH group; or
R₂ and R₃ together with the nitrogen atom to which they are attached form a 4- to 6-membered heterocycloalkyl;
in which R₇ is a —O—(C₁-C₆ alkyl) group or a —NH—N═CR₈-pyridyl group;
in which R₈ is a C₁-C₆ alkyl group;
more particularly,
R₂ and R₃ represents hydrogen, or a C₁-C₆ alkyl group; or
either of R₂ and R₃ represents H, the other of R₂ and R₃ represents a C₁-C₆ alkyl group, a C₂-C₆ alkenyl group, a C₃-C₆ cycloalkyl group, —NR₅R₆, pyridyl group, furyl group, phenyl group, a (C₁-C₆ alkyl)-pyridyl group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OH group; or
R₂ and R₃ together with the nitrogen atom to which they are attached form a 4- to 6-membered heterocycloalkyl;
in which R₇ is a —O—(C₁-C₆ alkyl) group or a —NH—N═CR₈-pyridyl group;
in which R₈ is a C₁-C₆ alkyl group;
additionally more particularly,
R₂ and R₃ represents hydrogen, or a C₁-C₆ alkyl group; or
either of R₂ and R₃ represents H, the other of R₂ and R₃ represents a C₁-C₆ alkyl group, a C₂-C₆ alkenyl group, a C₃-C₆ cycloalkyl group, —NR₅R₆, pyridin-2-yl group, pyridin-3-yl group, furan-2-yl group, phenyl group, a (C₁-C₆ alkyl)-(pyridin-2-yl) group, a (C₁-C₆ alkyl)-phenyl group, a (C₁-C₆ alkyl)-CO—R₇ group, a (C₁-C₆ alkyl)-NR₅R₆ group, or a (C₁-C₆ alkyl)-OH group; or
R₂ and R₃ together with the nitrogen atom to which they are attached form a 4- to 6-membered heterocycloalkyl;
in which R₇ is a —O—(C₁-C₆ alkyl) group or a —NH—N═CR₈-(pyridin-2-yl) group;
in which R₈ is a C₁-C₆ alkyl group.

6. The compounds according to claim 5, selected from the group consisting of
(E)-N′-(1-(pyridin-2-yl)ethylidene)azetidine-1-carbohydrazide,
(E)-N′-(2,2-dimethyl-1-(pyridin-2-yl)propylidene)azetidine-1-carbothiohydrazide,
(E)-N′-(pyridin-2-ylmethylene)azetidine-1-carbothiohydrazide,
(E)-N′-(1-(pyridin-2-yl)propylidene)azetidine-1-carbothiohydrazide,
(E)-N′-(1-(pyridin-3-yl)ethylidene)azetidine-1-carbothiohydrazide,
(E)-N′-(1-(pyridin-4-yl)ethylidene)azetidine-1-carbothiohydrazide,
(E)-N′-(phenyl (pyridin-2-yl)methylene)azetidine-1-carbothiohydrazide,
(E)-N′-(1-(1H-pyrrol-2-yl)ethylidene)azetidine-1-carbothiohydrazide,
N′-(propan-2-ylidene)azetidine-1-carbothiohydrazide,
(E)-N′-(1-(2-hydroxyphenyl)ethylidene)azetidine-1-carbothiohydrazide,
(E)-2-(1-(pyridin-2-yl)ethylidene)hydrazine-1-carbothioamide,
(E)-N,N-dimethyl-2-(1-(pyridin-2-yl)ethylidene)hydrazine-1-carbothioamide,
(E)-N,N-diethyl-2-(1-(pyridin-2-yl)ethylidene)hydrazine-1-carbothioamide,
(E)-N′-(1-(pyridin-2-yl)ethylidene)piperidine-1-carbothiohydrazide,
(E)-N-phenyl-2-(1-(pyridin-2-yl)ethylidene)hydrazine-1-carbothioamide,
(E)-N-(pyridin-2-yl)-2-(1-(pyridin-2-yl)ethylidene)hydrazine-1-carbothioamide,
(E)-N-propyl-2-(1-(pyridin-2-yl)ethylidene)hydrazine-1-carbothioamide,
(E)-N-benzyl-2-(1-(pyridin-2-yl)ethylidene)hydrazine-1-carbothioamide,
(E)-N-cyclohexyl-2-(1-(pyridin-2-yl)ethylidene)hydrazine-1-carbothioamide,
(E)-N′-(1-(pyridin-2-yl)ethylidene)hydrazinecarbothiohydrazide,
2-(propan-2-ylidene)hydrazine-1-carbothioamide,
(E)-2-(1-(thiophen-2-yl)ethylidene)hydrazine-1-carbothioamide,
(E)-2-(1-(pyridin-2-yl)propylidene)hydrazine-1-carbothioamide,
(E)-2-(1-(furan-2-yl)ethylidene)hydrazine-1-carbothioamide,
(E)-2-(cyclopropyl (pyridin-2-yl)methylene)hydrazine-1-carbothioamide,
(E)-N′-(1-(thiophen-2-yl)ethylidene)hydrazinecarbothiohydrazide,
(E)-N′-(1-(furan-2-yl)ethylidene)hydrazinecarbothiohydrazide,
(E)-N-(pyridin-2-yl)-2-(1-(thiophen-2-yl)ethylidene)hydrazine-1-carbothioamide,
(E)-2-(1-(furan-2-yl)ethylidene)-N-(pyridin-2-yl)hydrazine-1-carbothioamide,
(E)-N-methyl-2-(1-(pyridin-2-yl)ethylidene)hydrazine-1-carbothioamide,
(E)-N-allyl-2-(1-(pyridin-2-yl)ethylidene)hydrazine-1-carbothioamide,
(E)-N-cyclopropyl-2-(1-(pyridin-2-yl)ethylidene)hydrazine-1-carbothioamide,
(E)-N-(2-(dimethylamino)ethyl)-2-(1-(pyridin-2-yl)ethylidene)hydrazine-1-carbothioamide,
(E)-N-(furan-2-ylmethyl)-2-(1-(pyridin-2-yl)ethylidene)hydrazine-1-carbothioamide,
(E)-2-(1-(pyridin-2-yl)ethylidene)-N-(pyridin-2-ylmethyl)hydrazine-1-carbothioamide,
(E)-2-(1-(pyridin-2-yl)ethylidene)-N-(pyridin-3-yl)hydrazine-1-carbothioamide,
(E)-N-(2-hydroxyethyl)-2-(1-(pyridin-2-yl)ethylidene)hydrazine-1-carbothioamide,
Ethyl (E)-(2-(1-(pyridin-2-yl)ethylidene)hydrazine-1-carbonothioyl)glycinate, and
(E)-N-(2-oxo-2-(2- ((E)-1-(pyridin-2-yl)ethylidene)hydrazinyl)ethyl)-2-(1-(pyridin-2-yl)ethylidene)hydrazine-1-carbothioamide;
preferably,
(E)-N′-(pyridin-2-ylmethylene)azetidine-1-carbothiohydrazide,
(E)-N′-(1-(pyridin-2-yl)propylidene)azetidine-1-carbothiohydrazide,
(E)-N′-(phenyl (pyridin-2-yl)methylene)azetidine-1-carbothiohydrazide,
(E)-2-(1-(pyridin-2-yl)ethylidene)hydrazine-1-carbothioamide,
(E)-N,N-dimethyl-2-(1-(pyridin-2-yl)ethylidene)hydrazine-1-carbothioamide,
(E)-N,N-diethyl-2-(1-(pyridin-2-yl)ethylidene)hydrazine-1-carbothioamide,
(E)-N′-(1-(pyridin-2-yl)ethylidene)piperidine-1-carbothiohydrazide,
(E)-N-phenyl-2-(1-(pyridin-2-yl)ethylidene)hydrazine-1-carbothioamide,
(E)-N-(pyridin-2-yl)-2-(1-(pyridin-2-yl)ethylidene)hydrazine-1-carbothioamide,
(E)-N-propyl-2-(1-(pyridin-2-yl)ethylidene)hydrazine-1-carbothioamide,
(E)-N-benzyl-2-(1-(pyridin-2-yl)ethylidene)hydrazine-1-carbothioamide,
(E)-N-cyclohexyl-2-(1-(pyridin-2-yl)ethylidene)hydrazine-1-carbothioamide,
(E)-N′-(1-(pyridin-2-yl)ethylidene)hydrazinecarbothiohydrazide,
(E)-N-methyl-2-(1-(pyridin-2-yl)ethylidene)hydrazine-1-carbothioamide,
(E)-N-allyl-2-(1-(pyridin-2-yl)ethylidene)hydrazine-1-carbothioamide,
(E)-N-cyclopropyl-2-(1-(pyridin-2-yl)ethylidene)hydrazine-1-carbothioamide,
(E)-N-(2-(dimethylamino)ethyl)-2-(1-(pyridin-2-yl)ethylidene)hydrazine-1-carbothioamide,
(E)-N-(furan-2-ylmethyl)-2-(1-(pyridin-2-yl)ethylidene)hydrazine-1-carbothioamide,
(E)-2-(1-(pyridin-2-yl)ethylidene)-N-(pyridin-2-ylmethyl)hydrazine-1-carbothioamide,
(E)-N-(2-hydroxyethyl)-2-(1-(pyridin-2-yl)ethylidene)hydrazine-1-carbothioamide,
Ethyl (E)-(2-(1-(pyridin-2-yl)ethylidene)hydrazine-1-carbonothioyl)glycinate, and
(E)-N-(2-oxo-2-(2- ((E)-1-(pyridin-2-yl)ethylidene)hydrazinyl)ethyl)-2-(1-(pyridin-2-yl)ethylidene)hydrazine-1-carbothioamide;
more preferably,
(E)-N′-(1-(pyridin-2-yl)ethylidene)hydrazinecarbothiohydrazide.

7. The compounds according to claim 1, for treatment and/or prevention of neurodegeneration and age-associated diseases or conditions associated with NAD loss, especially amyotrophic lateral sclerosis, Parkinson's disease, traumatic brain injury, neonatal nerve crush injury, Alzheimer's disease, Chemotherapy-induced peripheral neuropathy (CIPN), ischemia, retinal degeneration, age-associated deficiency of neurogenesis, hypoadiponectinemia, and multi-organ insulin resistance.

8. A pharmaceutical preparation comprising at least one compound as defined in claim 1 in combination with an inert, non-toxic, pharmaceutically suitable excipient.

9. A method for treating and/or preventing neurodegeneration and age-associated diseases or conditions associated with NAD loss, especially amyotrophic lateral sclerosis, Parkinson's disease, traumatic brain injury, neonatal nerve crush injury, Alzheimer's disease, Chemotherapy-induced peripheral neuropathy (CIPN), ischemia, retinal degeneration, age-associated deficiency of neurogenesis, hypoadiponectinemia, and multi-organ insulin resistance in a subject, comprising administering at least one compound according to claim 1 to said subject.

10. A method for preparing the compound as defined in claim 1, comprising scheme 5 and any of schemes 1 to 4, 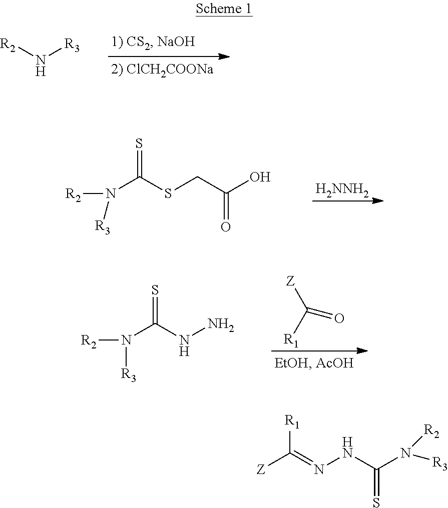
in which groups Z, R₁, R₂, and R₃ are defined in any of claims 1 to 6; 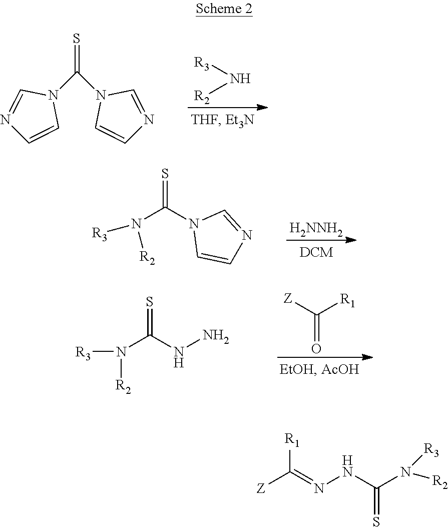
in which groups Z, R₁, R₂, and R₃ are defined in any of claims 1 to 6; 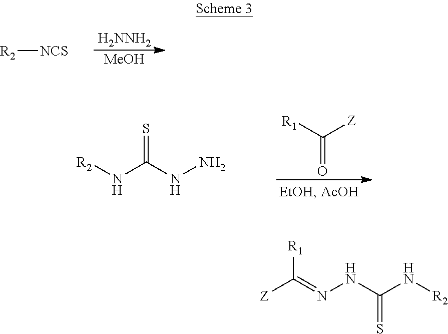
in which groups Z, R₁, and R₂ are defined in any of claims 1 to 6; 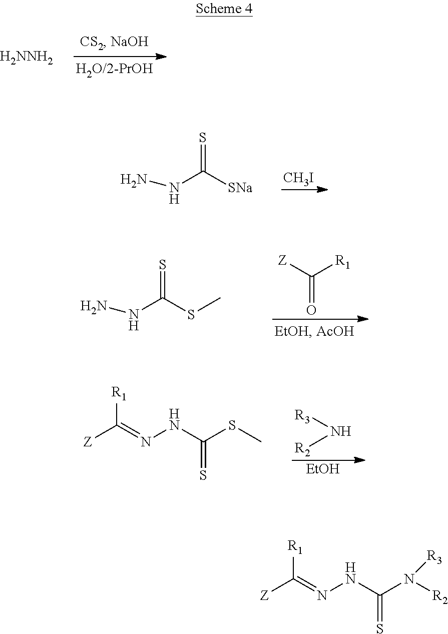
in which groups Z, R₁, R₂, and R₃ are defined in any of claims 1 to 6; 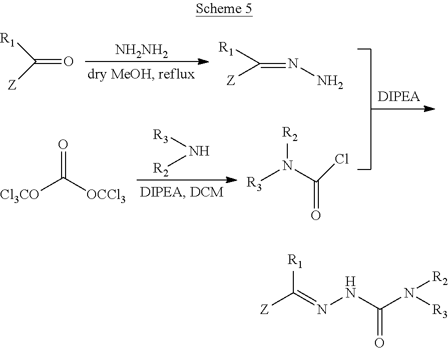
in which groups Z, R₁, R₂, and R₃ are defined in any of claims 1 to 6.

11. A method for high throughput screening of NMNAT2 activators, comprising
(a) adding compounds to NMNAT2 enzyme reaction mixture;
(b) initiating NMNAT2 enzyme reaction by adding NMN;
(c) monitoring the conversion of NMN to NADH by measuring fluorescence intensity of NADH at an excitation of 340 nm and an emission of 445 nm (Y axis) as a function of time (X axis), and then calculating reaction rate;
(d) obtaining relative NMNAT2 enzyme reaction rate in the presence of each compound by normalizing with DMSO-treated control; and
(e) selecting compounds with the relative reaction rate higher than 120% as NMNAT2 activators.

12. The method according to claim 11, further comprising
(f) selecting and re-testing the compounds with the relative reaction rate higher than 120% for ADH enzyme assay;
(g) adding the selected compounds to ADH enzyme reaction mixture;
(h) initiating ADH enzyme reaction by adding NAD;
(i) monitoring the conversion of NAD to NADH by measuring fluorescence intensity of NADH at an excitation of 340 nm and an emission of 445 nm (Y axis) as a function of time (X axis), and calculating reaction rate;
(j) obtaining relative ADH enzyme reaction rate in the presence of each compound by normalizing with DMSO-treated control; and
(k) selecting compounds that are active (relative reaction rate higher than 120%) in NMNAT2 enzyme assay but inactive (relative reaction rate lower than or equivalent to 100%) in the ADH enzyme assay as NMNAT2 activators.
